SEQUENCE LISTING (55 sequences)

SEQ ID NO: 1 - protein, 701 aa, synthetic construct
MSWKVVIIFSLLITPQHGLKESYLEESCSTITEGYLSVLRTGWYTNVFTLEVGDVENLTCADGPSLIKTELDLTKSALRELRTVSADQLAREEQIEGGGGGGFVLGAIALGVATAAAVTAGVAIAKCIRLESEVTAIKNALKKTNEAVSTLGCGVRVLATAVRELKDFVSKNLTRAINKNKCDIPDLKMAVSFSQFNRRFLNVVRQFSDNAGITPAISKDLMTDAELARAISNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSCGRNPISMVALSPLGALVACYKGVSCSIGSNRVGIIKQLNKGCSYITNQDADTVTIDNTVYQLSKVEGEQHVIKGRPVSSSFDPVKFPEDQFNVALDQCFESIENSQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 2 - protein, 447 aa, synthetic construct
QVQLQQPGAELVKPGASVKMSCKASGYTFTSYWITWVKQRPGQGLEWIGDIYPGSGSITYNEKFKTKATLTVDTSSSTAYMQLNSLTSEDSAVYYCAGLLRLTFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 7 aa, synthetic construct
GYTFTSY

SEQ ID NO: 4 - protein, 6 aa, synthetic construct
YPGSGS

SEQ ID NO: 5 - protein, 8 aa, synthetic construct
LLRLTFDV

SEQ ID NO: 6 - protein, 214 aa, synthetic construct
DIQLTQTTSSLSASLGDRVTISCRASQDISNYLNWYQQKPDGTVKLLISYTSGLHSGVPSRFSGSGSGTDYSLTISNLEQEDIATYFCQQGNTLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 11 aa, synthetic construct
RASQDISNYLN

SEQ ID NO: 8 - protein, 7 aa, synthetic construct
YTSGLHS

SEQ ID NO: 9 - protein, 9 aa, synthetic construct
QQGNTLPWT

SEQ ID NO: 10 - protein, 453 aa, synthetic construct
EVKLVESGGGLVQPGGSLSLSCAASGFTFTDYYMNWVRQPPGKALEWLGFIRNKDNGYTTEYSASVKGRFTISRDNSQSILYLQMNALRAEDSATYYCTRYYFGYDGDYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 7 aa, synthetic construct
GFTFTDY

SEQ ID NO: 12 - protein, 8 aa, synthetic construct
RNKDNGYT

SEQ ID NO: 13 - protein, 12 aa, synthetic construct
YYFGYDGDYFDY

SEQ ID NO: 14 - protein, 215 aa, synthetic construct
EIVLTQSPTTMAASPGEKITITCSASSSISSNYLHWYQQKAGFSPKLLIYRTSNLASGVPARFSGSGSGTSYSLTIGTMEAEDVATYYCQQGSSLPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 12 aa, synthetic construct
SASSSISSNYLH

SEQ ID NO: 16 - protein, 7 aa, synthetic construct
RTSNLAS

SEQ ID NO: 17 - protein, 9 aa, synthetic construct
QQGSSLPRT

SEQ ID NO: 18 - protein, 451 aa, synthetic construct
QVTLKESGPGILQPSQTLSLTCSFSGFSLSTFGMGVVWTRQPSGKGLEWLAHIWWDDDNYYNPALKSRLTISKDTSKNQVFLKIANADTADTATYYCARIVKVLEQYFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 9 aa, synthetic construct
GFSLSTFGM

SEQ ID NO: 20 - protein, 5 aa, synthetic construct
WWDDD

SEQ ID NO: 21 - protein, 11 aa, synthetic construct
IVKVLEQYFDV

SEQ ID NO: 22 - protein, 214 aa, synthetic construct
DIMMTQSHKFMSTSVGDRVIFTCKASQDVGTAVAWYQQKSGQSPKLLIYWASTRHTGVPDRFTGSGSGTDFTLTISNVQSEDLADYFCQQYTSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 11 aa, synthetic construct
KASQDVGTAVA

SEQ ID NO: 24 - protein, 7 aa, synthetic construct
WASTRHT

SEQ ID NO: 25 - protein, 9 aa, synthetic construct
QQYTSYPLT

SEQ ID NO: 26 - DNA, 549 bp, synthetic construct
  source 1..549: mol_type = other DNA
atggaaaacaaaatggattttcagatgcagattatcagcttgctgctaatcagtgtcacagtcatagtgtctactggagaaattgtgctcacccagtctccgaccaccatggctgcatctcccggggagaagatcactatcacctgcagtgccagctcaagtataagttccaattacttacattggtatcagcagaaggcaggattctcccctaaactcttgatttataggacatccaatctggcttctggagtcccagctcgcttcagtggcagtgggtctgggacctcttactctctcacaattggcaccatggaggctgaagatgttgccacttactactgccagcagggtagtagtctaccacgcacgttcggaggggggaccaagctggaaataaaacgggctgatgctgcaccaactgtatccatcttcccaccatccagtgagcagttaacatctggaggtgcctcagtcgtgtgcttcttgaacaacttctaccccagagacatcaatgtcaagtggaagattgatggcagtgaacgacaa

SEQ ID NO: 27 - protein, 26 aa, synthetic construct
MENKMDFQMQIISLLLISVTVIVSTG

SEQ ID NO: 28 - protein, 108 aa, synthetic construct
EIVLTQSPTTMAASPGEKITITCSASSSISSNYLHWYQQKAGFSPKLLIYRTSNLASGVPARFSGSGSGTSYSLTIGTMEAEDVATYYCQQGSSLPRTFGGGTKLEIK

SEQ ID NO: 29 - protein, 49 aa, synthetic construct
RADAAPTVSIFPPSSEQLTSGGASVVCFLNNFYPRDINVKWKIDGSERQ

SEQ ID NO: 30 - DNA, 591 bp, synthetic construct
  source 1..591: mol_type = other DNA
atgaagttgtggctgaactggattttccttgtaacacttttaaatggtatccagtgtgaggtgaagctggtggagtctggaggaggcttggtacagcctgggggttctctgagtctctcctgtgcagcttctggattcaccttcactgattactacatgaactgggtccgccagcctccagggaaggcacttgagtggttgggctttattagaaacaaagataatggttacacaacagagtatagtgcatctgtgaagggtcggttcaccatctccagagataattcccaaagcatcctctatcttcaaatgaatgccctgagagctgaggacagtgccacttattactgtacaagatattactttggttacgacggggactactttgactactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagct

SEQ ID NO: 31 - protein, 19 aa, synthetic construct
MKLWLNWIFLVTLLNGIQC

SEQ ID NO: 32 - protein, 123 aa, synthetic construct
EVKLVESGGGLVQPGGSLSLSCAASGFTFTDYYMNWVRQPPGKALEWLGFIRNKDNGYTTEYSASVKGRFTISRDNSQSILYLQMNALRAEDSATYYCTRYYFGYDGDYFDYWGQGTTLTVSS

SEQ ID NO: 33 - protein, 55 aa, synthetic construct
AKTTPPSVYPLAPGSAAQTNSMVTLGCLVKGYFPEPVTVTWNSGSLSSGVHTFPA

SEQ ID NO: 34 - DNA, 525 bp, synthetic construct
  source 1..525: mol_type = other DNA
atgtcctctgctcagttccttggtctcctgttgctctgttttcaaggtgccagatgtgatatccagctgacacagactacatcctccctgtctgcctctctgggagacagagtcaccatcagttgcagggcaagtcaggacattagcaattatttaaactggtatcagcagaaaccagatggaactgttaaactcctgatctcctacacatcaggattacactcaggagtcccatcaaggttcagtggcagtgggtctggaacagattattctctcaccattagcaacctggagcaagaagatattgccacttacttttgccaacagggtaatacgcttccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccagagacatcaatgtcaagtggaagattgatggcagtgaacgacaa

SEQ ID NO: 35 - protein, 19 aa, synthetic construct
MSSAQFLGLLLLCFQGARC

SEQ ID NO: 36 - protein, 107 aa, synthetic construct
DIQLTQTTSSLSASLGDRVTISCRASQDISNYLNWYQQKPDGTVKLLISYTSGLHSGVPSRFSGSGSGTDYSLTISNLEQEDIATYFCQQGNTLPWTFGGGTKLEIK

SEQ ID NO: 37 - protein, 49 aa, synthetic construct; identical to SEQ ID NO: 29

SEQ ID NO: 38 - DNA, 573 bp, synthetic construct
  source 1..573: mol_type = other DNA
atgggatggagctgtatcatcctcattttggtagcagcagctacaggtgtccactcccaggtccaactgcagcagcctggggctgagcttgtgaagcctggggcttcagtgaagatgtcctgcaaggcttctggctacaccttcaccagctactggataacctgggtgaagcagaggcctggacagggccttgagtggattggagatatttatcctggtagtggtagtattacctacaatgagaagttcaagaccaaggccacactgactgtagacacatcctccagcacagcctacatgcagctcaacagcctgacatctgaggactctgcggtctattactgtgcaggattactacggttaaccttcgatgtctggggcacagggaccacggtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagct

SEQ ID NO: 39 - protein, 19 aa, synthetic construct
MGWSCIILILVAAATGVHS

SEQ ID NO: 40 - protein, 117 aa, synthetic construct
QVQLQQPGAELVKPGASVKMSCKASGYTFTSYWITWVKQRPGQGLEWIGDIYPGSGSITYNEKFKTKATLTVDTSSSTAYMQLNSLTSEDSAVYYCAGLLRLTFDVWGTGTTVTVSS

SEQ ID NO: 41 - protein, 55 aa, synthetic construct; identical to SEQ ID NO: 33

SEQ ID NO: 42 - DNA, 525 bp, synthetic construct
  source 1..525: mol_type = other DNA
atggagacacattctcaggtctttgtatacatgttgctgtggttgtctggtgttgaaggagacattatgatgacccagtctcacaaattcatgtccacatcagtaggagacagggtcattttcacctgcaaggccagtcaggatgtgggtactgctgtagcctggtatcaacagaaatcagggcaatctcctaaactactgatttactgggcatccacccggcacactggagtccctgatcgcttcacaggcagtggatctgggacagatttcactctcaccattagcaatgtgcagtctgaagacttggcagattatttctgtcaacaatataccagctatcctctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccagagacatcaatgtcaagtggaagattgatggcagtgaacga

SEQ ID NO: 43 - protein, 20 aa, synthetic construct
METHSQVFVYMLLWLSGVEG

SEQ ID NO: 44 - protein, 107 aa, synthetic construct
DIMMTQSHKFMSTSVGDRVIFTCKASQDVGTAVAWYQQKSGQSPKLLIYWASTRHTGVPDRFTGSGSGTDFTLTISNVQSEDLADYFCQQYTSYPLTFGAGTKLELK

SEQ ID NO: 45 - protein, 48 aa, synthetic construct
RADAAPTVSIFPPSSEQLTSGGASVVCFLNNFYPRDINVKWKIDGSER

SEQ ID NO: 46 - DNA, 570 bp, synthetic construct
  source 1..570: mol_type = other DNA
atgggcaggcttacttcttcattcctgttactgattgtccctgcatatgtcctgtcccaggttactctgaaagagtctggccctgggatattgcagccctcccagaccctcagtctgacttgttctttctctgggttttcactgagcacttttggtatgggtgtagtctggactcgtcagccttcagggaagggtctggagtggctggcacacatttggtgggatgatgataattactataacccagccctgaagagtcggctcacaatctccaaggatacctccaaaaaccaggtattcctcaaaatcgccaatgcggacactgcagatactgccacatactactgtgctcgaatagttaaggtcctggaacagtacttcgatgtctggggcacagggaccacggtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagtggtgtg

SEQ ID NO: 47 - protein, 19 aa, synthetic construct
MGRLTSSFLLLIVPAYVLS

SEQ ID NO: 48 - protein, 121 aa, synthetic construct
QVTLKESGPGILQPSQTLSLTCSFSGFSLSTFGMGVVWTRQPSGKGLEWLAHIWWDDDNYYNPALKSRLTISKDTSKNQVFLKIANADTADTATYYCARIVKVLEQYFDVWGTGTTVTVSS

SEQ ID NO: 49 - protein, 50 aa, synthetic construct
AKTTPPSVYPLAPGSAAQTNSMVTLGCLVKGYFPEPVTVTWNSGSLSSGV

SEQ ID NO: 50 - protein, 117 aa, synthetic construct; identical to SEQ ID NO: 40

SEQ ID NO: 51 - protein, 107 aa, synthetic construct; identical to SEQ ID NO: 36

SEQ ID NO: 52 - protein, 123 aa, synthetic construct; identical to SEQ ID NO: 32

SEQ ID NO: 53 - protein, 108 aa, synthetic construct; identical to SEQ ID NO: 28

SEQ ID NO: 54 - protein, 121 aa, synthetic construct; identical to SEQ ID NO: 48

SEQ ID NO: 55 - protein, 107 aa, synthetic construct; identical to SEQ ID NO: 44

(19) United States
(12) Patent Application Publication
(10) Pub. No.: US 2026/0092100 A1
(43) Pub. Date: Apr. 2, 2026

(54) ANTIBODIES AGAINST METAPNEUMOVIRUS FUSION (F) PROTEIN AND USES THEREOF

(71) Applicant: Icosavax, Inc., Seattle, WA (US)

(72) Inventor: Andrew L. FELDHAUS, Seattle, WA (US)

(21) Appl. No.: 19/245,194

(22) Filed: Jun. 20, 2025

Related U.S. Application Data
(63) Continuation of application No. PCT/US2023/085719, filed on Dec. 22, 2023.
(60) Provisional application No. 63/477,092, filed on Dec. 23, 2022.

(51) Int. Cl. C07K 16/11; A61K 39/00; A61P 31/14

(52) U.S. Cl. CPC C07K 16/11; A61P 31/14; A61K 2039/54; A61K 2039/55; C07K 2317/24; C07K 2317/33; C07K 2317/565; C07K 2317/76; C07K 2317/92

(57) ABSTRACT

Provided are epitope-specific antigen-binding molecules specific to a human metapneumovirus (hMPV) Fusion (F) protein, in which the antigen-binding molecule comprises a variable domain that contacts the hMPV F protein. The antigen-binding molecule specific to an hMPV F protein comprises a heavy chain and a light chain. Further provided are methods for detecting antibodies specific to an hMPV F protein, a method comprising: a.) contacting a biological sample with an hMPV F protein; and b.) contacting an epitope-specific antigen-binding molecule with the hMPV F protein.

### CROSS-REFERENCE TO RELATED APPLICATION

This application is a continuation of International Application No. PCT/US2023/085719, filed Dec. 22, 2023, which claims the benefit of U.S. Provisional Patent Application No. 63/477,092 filed Dec. 23, 2022, each of which is incorporated by reference herein in its entirety.

### INCORPORATION BY REFERENCE OF SEQUENCE LISTING

This application contains a Sequence Listing which has been submitted in .XML format via EFS-WEB and is hereby incorporated by reference in its entirety. Said .XML copy, created on Oct. 15, 2025 is named 061291-510C01US_SeqList_ST26.xml and is 69,510 bytes in size.

### FIELD

The disclosure relates generally to antibodies against metapneumovirus fusion (F) protein.

### BACKGROUND

Human metapneumovirus (hMPV) causes upper and lower respiratory disease in people of all ages. The symptoms can include cough, fever, nasal congestion, and shortness of breath. In some cases, hMPV infection can progress to severe disease, such as bronchitis or pneumonia.

hMPV is a single-stranded RNA virus in the paramyxovirus family, which also includes measles, mumps, and other respiratory tract infections such as respiratory syncytial virus (RSV). hMPV can be of two subtypes, A and B, that are identified by genotyping the G and F genes in the viral genome. The F gene encodes hMPV fusion glycoprotein (hMPV F protein) and may be used in treating or preventing an hMPV infection. However, to date there has been no approved treatment for hMPV in humans despite the clinical need.

There is therefore an unmet need for a treatment for hMPV.

### SUMMARY

Provided are epitope-specific antigen-binding molecules specific to an hMPV Fusion (F) protein, wherein the antigen-binding molecule comprises a variable domain that contacts the hMPV F protein at
i.) residues 287, 293, 296, 364, 376, 417 and 419;
ii.) residues 144, 160, 163, 188, 194 and 199; or
iii.) residues 44, 45, 49, 150, 156, 160, 229, 232 and 236 of the hMPV F protein amino acid sequence set forth in SEQ ID NO: 1.

In some embodiments, an antigen-binding molecule specific to an hMPV F protein comprises a heavy chain and a light chain, wherein the heavy chain comprises an H-CDR1, an H-CDR2, and an H-CDR3 wherein: H-CDR1 comprises the sequence GYTFTSY (SEQ ID NO: 3); H-CDR2 comprises the sequence YPGSGS (SEQ ID NO: 4); and H-CDR3 comprises the sequence LLRLTFDV (SEQ ID NO: 5); and wherein the light chain comprises an L-CDR1, an L-CDR2, and an L-CDR3, wherein: L-CDR1 comprises the sequence RASQDISNYLN (SEQ ID NO: 7); L-CDR2 comprises the sequence YTSGLHS (SEQ ID NO: 8); and L-CDR3 comprises the sequence QQGNTLPWT (SEQ ID NO: 9).

In some embodiments, an antigen-binding molecule comprises a variable domain that contacts the hMPV F protein at
i.) residues 287, 293, 296, 364, 376, 417 and 419;
ii.) residues 144, 160, 163, 188, 194 and 199; or
iii.) residues 44, 45, 49, 150, 156, 160, 229, 232 and 236 of the hMPV F protein amino acid sequence set forth in SEQ ID NO: 1.

In some embodiments, the antigen-binding molecule comprises a variable domain that contacts the hMPV F protein at any 1, any 2, any 3, any 4, any 5, any 6, or any 7 of residues 287, 293, 296, 364, 376, 417 and 419 of the hMPV F protein amino acid sequence set forth in SEQ ID NO: 1.

In some embodiments, the heavy chain comprises a sequence having at least 70%, at least 80%, at least 90%, at least 95%, at least 97%, at least 98%, at least 99%, or 100% sequence identity to SEQ ID NO: 2.

In some embodiments, the light chain comprises a sequence having at least 70%, at least 80%, at least 90%, at least 95%, at least 97%, at least 98%, at least 99%, or 100% sequence identity SEQ ID NO: 6.

In some embodiments, the heavy chain comprises a sequence having at least 70%, at least 80%, at least 90%, at least 95%, at least 97%, at least 98%, at least 99%, or 100% sequence identity to SEQ ID NO: 2, and the light chain comprises a sequence having at least 70%, at least 80%, at least 90%, at least 95%, at least 97%, at least 98%, at least 99%, or 100% sequence identity to SEQ ID NO: 6.

In some embodiments, the antigen-binding molecule comprises a variable heavy (VH) chain domain wherein the VH domain comprises a sequence having at least 70%, at least 80%, at least 90%, at least 95%, at least 97%, at least 98%, at least 99%, or 100% sequence identity to SEQ ID NO: 50, and a variable light (VL) chain domain wherein the VL domain comprises a sequence having at least 70%, at least 80%, at least 90%, at least 95%, at least 97%, at least 98%, at least 99%, or 100% sequence identity to SEQ ID NO: 51.

In some embodiments, the antigen-binding molecule comprises a variable domain that contacts the hMPV F protein at any 1, any 2, any 3, any 4, any 5, or any 6 of residues 144, 160, 163, 188, 194 and 199 of the hMPV F protein amino acid sequence set forth in SEQ ID NO: 1.

In some embodiments, the antigen-binding molecule comprises a heavy chain and a light chain, wherein the heavy chain comprises an H-CDR1, an H-CDR2, and an H-CDR3 wherein: H-CDR1 comprises the sequence GFTFTDY (SEQ ID NO: 11); H-CDR2 comprises the sequence RNKDNGYT (SEQ ID NO: 12); and H-CDR3 comprises the sequence YYFGYDGDYFDY (SEQ ID NO: 13); and wherein the light chain comprises an L-CDR1, an L-CDR2, and an L-CDR3, wherein: L-CDR1 comprises the sequence SASSSISSNYLH (SEQ ID NO: 15); L-CDR2 comprises the sequence RTSNLAS (SEQ ID NO: 16); and L-CDR3 comprises the sequence QQGSSLPRT (SEQ ID NO: 17).

In some embodiments, the heavy chain comprises a sequence having at least 70%, at least 80%, at least 90%, at least 95%, at least 97%, at least 98%, at least 99%, or 100% sequence identity to SEQ ID NO: 10.

In some embodiments, the light chain comprises a sequence having at least 70%, at least 80%, at least 90%, at least 95%, at least 97%, at least 98%, at least 99%, or 100% sequence identity SEQ ID NO: 14.

In some embodiments, the heavy chain comprises a sequence having at least 70%, at least 80%, at least 90%, at least 95%, at least 97%, at least 98%, at least 99%, or 100% sequence identity to SEQ ID NO: 10, and the light chain comprises a sequence having at least 70%, at least 80%, at least 90%, at least 95%, at least 97%, at least 98%, at least 99%, or 100% sequence identity to SEQ ID NO: 14.

In some embodiments, the antigen-binding molecule comprises a variable heavy (VH) chain domain wherein the VH domain comprises a sequence having at least 70%, at least 80%, at least 90%, at least 95%, at least 97%, at least 98%, at least 99%, or 100% sequence identity to SEQ ID NO: 52, and a variable light (VL) chain domain wherein the VL domain comprises a sequence having at least 70%, at least 80%, at least 90%, at least 95%, at least 97%, at least 98%, at least 99%, or 100% sequence identity to SEQ ID NO: 53.

In some embodiments, the antigen-binding molecule comprises a variable domain that contacts the hMPV F protein at any 1, any 2, any 3, any 4, any 5, any 6, any 7, any 8, or any 9 of residues 44, 45, 49, 150, 156, 160, 229, 232 and 236 of the hMPV F protein amino acid sequence set forth in SEQ ID NO: 1.

In some embodiments, the antigen-binding molecule comprises a heavy chain and a light chain, wherein the heavy chain comprises an H-CDR1, an H-CDR2, and an H-CDR3 wherein: H-CDR1 comprises the sequence GFSLSTFGM (SEQ ID NO: 19); H-CDR2 comprises the sequence WWDDD (SEQ ID NO: 20); and H-CDR3 comprises the sequence IVKVLEQYFDV (SEQ ID NO: 21); and wherein the light chain comprises an L-CDR1, an L-CDR2, and an L-CDR3, wherein: L-CDR1 comprises the sequence KASQDVGTAVA (SEQ ID NO: 23); L-CDR2 comprises the sequence WASTRHT (SEQ ID NO: 24); and L-CDR3 comprises the sequence QQYTSYPLT (SEQ ID NO: 25).

In some embodiments, the heavy chain comprises a sequence having at least 70%, at least 80%, at least 90%, at least 95%, at least 97%, at least 98%, at least 99%, or 100% sequence identity to SEQ ID NO: 18.

In some embodiments, the light chain comprises a sequence having at least 70%, at least 80%, at least 90%, at least 95%, at least 97%, at least 98%, at least 99%, or 100% sequence identity SEQ ID NO: 22.

In some embodiments, the heavy chain comprises a sequence having at least 70%, at least 80%, at least 90%, at least 95%, at least 97%, at least 98%, at least 99%, or 100% sequence identity to SEQ ID NO: 18, and the light chain comprises a sequence having at least 70%, at least 80%, at least 90%, at least 95%, at least 97%, at least 98%, at least 99%, or 100% sequence identity to SEQ ID NO: 22.

In some embodiments, the antigen-binding molecule comprises a variable heavy (VH) chain domain wherein the VH domain comprises a sequence having at least 70%, at least 80%, at least 90%, at least 95%, at least 97%, at least 98%, at least 99%, or 100% sequence identity to SEQ ID NO: 54, and a variable light (VL) chain domain wherein the VL domain comprises a sequence having at least 70%, at least 80%, at least 90%, at least 95%, at least 97%, at least 98%, at least 99%, or 100% sequence identity to SEQ ID NO: 55.

In some embodiments, the antigen-binding molecule is an immunoglobulin molecule.

In some embodiments, the immunoglobulin is an IgG1, IgG2, IgG3, or IgG4 molecule.

In some embodiments, the immunoglobulin is a humanized antibody.

Provided is a polynucleotide encoding an antigen-binding molecule of the present disclosure.

Provided is a pharmaceutical composition comprising an antigen-binding molecule of the present disclosure, and a pharmaceutically acceptable carrier, diluent, or excipient.

Provided is a method for detecting antibodies specific to an hMPV F protein, the method comprising:
a.) contacting a biological sample with an hMPV F protein; and
b.) contacting an epitope-specific antigen-binding molecule according to any one of claims 1-19 with the hMPV F protein.

In some embodiments, the hMPV F protein is coated on a microwell plate prior to contact with the biological sample or antigen-binding molecule.

In some embodiments, a half maximal effective concentration (EC50) of anti-hMPV F protein antibodies in the biological sample is determined by calculating the reciprocal dilution of biological sample at which 50% of the binding of the antigen-binding molecule is inhibited.

In some embodiments, the biological sample is serum.

Provided is a method of treating or preventing an hMPV infection in a subject in need thereof, wherein the method comprises administering to the subject an effective amount of an antigen-binding molecule according to the present disclosure.

In some embodiments, the subject has or is at risk of developing an hMPV infection.

In some embodiments, the subject is a mammal, optionally a human.

In some embodiments, the subject is susceptible to viral infection.

In some embodiments, the subject is an elderly subject.

In some embodiments, the antigen-binding molecule of the present disclosure is administered by intramuscular injection, intravenous injection, or subcutaneous injection.

### BRIEF DESCRIPTION OF THE DRAWINGS

FIG. 1 shows an illustrative hMPV F protein with neutralizing antigenic sites and epitopes of the 17D10, 13E10 and 42C2 antibodies. The regions containing the epitopes for 17D10, 13E10 and 42C2 are circled as well as antigenic sites for neutralizing antibodies denoted as II, III, IV, V, DS7, and 66-87.

FIGS. 2A-2C are illustrative diagrams of the three hMPV033 epitopes for 17D10 (FIG. 2A) (SEQ ID NOs: 59-61, with the displayed amino acid target sequence comprising amino acid residues 280-300 (SEQ ID NO: 56), 360-380 (SEQ ID NO: 57), and 410-420 (SEQ ID NO: 58)); the two hMPV033 epitopes for, 13E10 (FIG. 2B) (SEQ ID NOs: 64-65, with the displayed amino acid target sequence comprising amino acid residues 140-170 (SEQ ID NO: 62) and 180-200 (SEQ ID NO: 63));, and the three hMPV033 epitopes for 42C2 (FIG. 2C) (SEQ ID NOs 69-71, with the displayed amino acid target sequence comprising amino acid residues 40-50 (SEQ ID NO: 66), 150-160 (SEQ ID NO: 67), and 220-240 (SEQ ID NO: 68)).

FIGS. 3A-3C are graphs depicting binding of mouse (mIgG) and human (hIgG) Fc-containing neutralizing antibodies (nAbs) 17D10 (FIG. 3A), 13E10 (FIG. 3B), and 42C2 (FIG. 3C) to hMPV033 (top panels) and DS-Cav1 (RSV control) (bottom panels). All three nAbs were specific for hMPV F and not RSV F nor I53-50A.

FIGS. 4A-4J are diagrams depicting the interaction of hMPV033 and 17D10_hlgG1. hMPV033 Protein Data Bank (PDB) structure was colored in blue on the epitope sites. hMPV033 amino acids colored in blue correspond to amino acids 287-296 (KAAPSCSEKK) (SEQ ID NO: 59), 364-376 (SCGRNPISMVALS) (SEQ ID NO: 60), and 417-419 (TVT) (SEQ IF NO: 61) of hMPV033 sequence SEQ ID NO: 1. FIGS. 4A, 4B, 4C, 4D, and 4E show ribbon/surface representations of front view (FIG. 4A); back view (FIG. 4B), side view 1 (FIG. 4C), side view 2 (FIG. 4D) and top view (FIG. 4E). FIGS. 4F, 4G, 4H, 4I, 4J: show ribbon representations of front view (FIG. 4F); back view (FIG. 4G), side view 1 (FIG. 4H), side view 2 (FIG. 4I) and top view (FIG. 4J).

FIGS. 5A-5J are diagrams depicting the interaction of hMPV033 and 13E10_hlgG1. hMPV033 PDB structure was colored in blue on the epitope sites. hMPV033 amino acids colored in blue correspond to amino acids 144-163 (TNEAVSTLGCGVRVLATAVR) (SEQ ID NO: 64) and 188-199 (KMAVSFSQFNRR) (SEQ ID NO: 65) of hMPV033 sequence SEQ ID NO: 1. FIGS. 5A, 5B, 5C, 5D, and 5E show ribbon/surface representations of front view (FIG. 5A); back view (FIG. 5B), side view 1 (FIG. 5C), side view 2 (FIG. 5D), and top view (FIG. 5E). FIGS. 5F, 5G, 5H, 5I, and 5J show ribbon representations of front view (FIG. 5F); back view (FIG. 5G), side view 1 (FIG. 5H), side view 2 (FIG. 5I) and top view (FIG. 5J).

FIGS. 6A-6J are diagrams depicting the interaction of hMPV033 and 42C2_hlgG1. hMPV033 PDB structure was colored in blue on the epitope sites. hMPV033 amino acids colored in blue correspond to amino acids 44-49 (YTNVFT) (SEQ ID NO: 69), 150-160 (TLGCGVRVLAT) (SEQ ID NO: 70), and 229-236 (RAISNMPT) (SEQ ID NO: 71) of hMPV033 sequence SEQ ID NO: 1. FIGS. 6A, 6B, 6C, 6D, and 6E show ribbon/surface representations of front view (FIG. 6A); back view (FIG. 6B), side view 1 (FIG. 6C), side view 2 (FIG. 6D), and top view (FIG. 6E). FIGS. 6F, 6G, 6H, 6I, and 6J show ribbon representations of front view (FIG. 6F); back view (FIG. 6G), side view 1 (FIG. 6H), side view 2 (FIG. 6I) and top view (FIG. 6J).

FIGS. 7A-7D are graphs depicting binding of antibodies to pre- and post-fusion hMPV F protein. Binding of MF1, MF2, and MF3 post-fusion hMPV F protein specific antibodies and MF10 pre-fusion hMPV F protein specific antibody (control) were analyzed by Octet for binding to pre-fusion hMPV F protein (FIG. 7A) or post-fusion hMPV F protein (FIG. 7B). Binding of 17D10, 42C2 and 13E10 hMPV F protein specific antibodies were analyzed by Octet for binding to pre-fusion hMPV F protein (FIG. 7C) or post-fusion hMPV F protein (FIG. 7D). 17D10 and 42C2 antibodies bind both pre- and post-fusion conformations of hMPV F protein; 13E10 binds only the pre-fusion hMPV F protein conformation.

### DETAILED DESCRIPTION

### Definitions

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described. For the purposes of the present disclosure, the following terms are defined below.

The term “antigen” refers to a polypeptide or polypeptide complex including at least one component designed to elicit an immune response. The term antigen, as used herein, is not limited to the portion of the polypeptide or polypeptide complex that contains antigenic epitopes.

The term “infection” refers to both symptomatic and asymptomatic infections.

The term “linker” refers to either chemical linkage (i.e., a covalent bond or series of covalent bonds with intervening chemical moieties) or to a polypeptide that is N-terminally and C-terminally joined by peptide bonds to produce a fusion protein.

The term “antigen-binding molecule” refers to a molecule that has binding affinity for a target antigen. It will be understood that this term extends to immunoglobulins, immunoglobulin fragments, and non-immunoglobulin derived protein frameworks that exhibit antigen-binding activity. Representative antigen-binding molecules that are useful in the practice of the present disclosure include polyclonal and monoclonal antibodies as well as their fragments (such as Fab, Fab′, F(ab′)₂, Fv), single chain (scFv) and domain antibodies (including, for example, shark and camelid antibodies), and fusion proteins comprising an antibody, and any other modified configuration of the immunoglobulin molecule that comprises an antigen binding/recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called α, δ, ε, γ, and μ, respectively. The subunit structures and three-dimensional configurations of different classes of immunoglobulins are well known. Antigen-binding molecules also encompass dimeric antibodies, as well as multivalent forms of antibodies. In some embodiments, the antigen-binding molecules are chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, for example, U.S. Pat. No. 4,816,567; and Morrison et al., 1984, Proc. Natl. Acad. Sci. USA 81:6851-6855). Also contemplated, are humanized antibodies, which are generally produced by transferring complementarity determining regions (CDRs) from heavy and light variable chains of a non-human (e.g., rodent, preferably mouse) immunoglobulin into a human variable domain. Typical residues of human antibodies are then substituted in the framework regions of the non-human counterparts. The use of antibody components derived from humanized antibodies obviates potential problems associated with the immunogenicity of non-human constant regions. General techniques for cloning non-human, particularly murine, immunoglobulin variable domains are described, for example, by Orlandi et al. (1989, Proc. Natl. Acad. Sci. USA 86: 3833). Techniques for producing humanized monoclonal antibodies are described, for example, by Jones et al. (1986, Nature 321:522), Carter et al. (1992, Proc. Natl. Acad. Sci. USA 89: 4285), Sandhu (1992, Crit. Rev. Biotech. 12: 437), Singer et al. (1993, J. Immun. 150: 2844), Sudhir (ed., Antibody Engineering Protocols, Humana Press, Inc. 1995), Kelley (“Engineering Therapeutic Antibodies,” in Protein Engineering: Principles and Practice), Cleland et al. (eds.), pages 399-434 (John Wiley & Sons, Inc. 1996), and by Queen et al., U.S. Pat. No. 5,693,762 (1997).

The term “antibody” as used herein is used in the broadest sense and specifically covers naturally occurring antibodies, monoclonal antibodies, polyclonal antibodies, multi-specific antibodies (e.g., bispecific antibodies), and antibody fragments, which exhibit the desired immuno-interactivity. A naturally occurring “antibody” includes within its scope an immunoglobulin comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of specific CH domains (e.g., CH1, CH2 and CH3). Each light chain is comprised of a light chain variable region (abbreviated herein as VL) and a light chain constant region. The light chain constant region is comprised of one domain, CL. The VH and VL regions can be further subdivided into regions of hypervariability, termed complementary determining regions (CDR). Each VH and VL is composed of three CDRs. The antibodies can be of any isotype (e.g., IgG, IgE, IgM, IgD, IgA and IgY), class (e.g., IgG1, IgG2, IgG3, IgG4, IgA1 and IgA2), subclass or modified version thereof (e.g., IgG1 isotype, which carries L234A and L235A double mutations (IgG1-LALA)). The antibodies can be of any species, chimeric, humanized, or human. In other embodiments, the antibody is a homomeric heavy chain antibody (e.g., camelid antibodies) which lacks the first constant region domain (CH1) but retains an otherwise intact heavy chain and is able to bind antigens through an antigen-binding domain. Unless otherwise indicated, the term “antibody” includes, in addition to antibodies comprising two full-length heavy chains and two full-length light chains, derivatives, variants, fragments, and muteins thereof, examples of which are described below. Furthermore, unless explicitly excluded, antibodies include monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), and fragments thereof, respectively. In some embodiments, the term also encompasses peptibodies.

In certain embodiments, an antibody heavy chain binds to an antigen in the absence of an antibody light chain. In certain embodiments, an antibody light chain binds to an antigen in the absence of an antibody heavy chain. In certain embodiments, an antibody binding region binds to an antigen in the absence of an antibody light chain. In certain embodiments, an antibody binding region binds to an antigen in the absence of an antibody heavy chain. In certain embodiments, an individual variable region specifically binds to an antigen in the absence of other variable regions.

The term “neutralizing” (e.g., “neutralizing antibody”) refers to antibodies that prevent infection and/or reduce the level of infection by a pathogen. A neutralizing antibody response can be measured either in an in vitro assay (e.g., infection of cells in culture by a pathogen in the presence of the antibody) or in an in vivo assay (e.g., by determining a protective dose of an antibody through administering the antibody to a subject prior to challenge with an infective dose of a pathogen). Neutralizing antibodies can inhibit pathogen infectivity by binding to the pathogen and blocking the molecules needed for host cell entry. Neutralizing antibodies can statically interfere with the pathogens attachment to host cell receptors. Without being bound by theory, in the case of a virus infection, neutralizing antibodies can bind to glycoproteins of enveloped viruses or capsid proteins of non-enveloped viruses and can act by preventing viral particles from undergoing structural changes often needed for successful host cell entry.

The terms “variable region” or “variable domain” refer to a variable domain of a light chain (VL) or a variable domain of a heavy chain (VH). As used herein, “variable region” or “variable domain” denote each of the pair of light and heavy chain domains which are involved directly in binding the antibody to the antigen. The variable light and heavy chain domains have the same general structure, and each domain comprises four framework regions (FRs) whose sequences are widely conserved, connected by three CDRs or “hypervariable regions”. The FRs adopt a β-sheet conformation and the CDRs may form loops connecting the p-sheet structure. The CDRs in each chain are held in their three-dimensional structure by the FRs which form together with the CDRs to form the antigen binding site.

The “CDR” or “complementarity determining region” (also referred to as “hypervariable region”) are the amino acid residues of an antibody which are responsible for antigen-binding. As used herein, CDRs refer to the amino acid sequences of the light and heavy chains of an antibody which form the three-dimensional loop structure that contributes to the formation of an antigen binding site. There are three CDRs, which are designated “CDR1”, “CDR2”, and “CDR3”, in each of the variable regions of the heavy chain and the light chain of an antibody. The term “CDR set” as used herein refers to a group of three CDRs that occur in a single variable region that binds the antigen. The terms “heavy chain variable region CDR1” and “H-CDR1” are used interchangeably, as are the terms “heavy chain variable region CDR2” and “H-CDR2”, “heavy chain variable region CDR3” and “H-CDR3”, “light chain variable region CDR1” and “L-CDR1”, “light chain variable region CDR2” and “L-CDR2”, and “light chain variable region CDR3” and “L-CDR3”.

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. Examples of such methods include, but are not limited to, the Kabat definition, the Chothia definition, the AbM definition and the contact definition.

The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as “Kabat CDRs”. Chothia and coworkers (Chothia and Lesk, 1987. J. Mol. Biol. 196: 901-917; Chothia et al., 1989. Nature 342: 877-883) found that certain sub-portions within Kabat CDRs adopt nearly identical peptide backbone conformations, despite having great diversity at the level of amino acid sequence. Other boundaries defining CDRs overlapping with the Kabat CDRs have been described by Padlan (1995. FASEB J. 9: 133-139) and MacCallum (1996. J Mol. Biol. 262(5): 732-745). Still other CDR boundary definitions may not strictly follow one of these systems, but will nonetheless overlap with the Kabat CDRs, although they may be shortened or lengthened in light of prediction or experimental findings that particular residues or groups of residues or even entire CDRs do not significantly impact antigen binding.

A “single chain variable Fragment (scFv)” is a single protein chain in which the VL and VH regions pair to form monovalent molecules (known as single chain Fv (scFv); see, e.g., Bird et al., 1988. Science 242:423-426; and Huston et al., 1988. Proc. Natl. Acad. Sci. 85:5879-5883). Although the two domains VL and VH are coded for by separate genes, they can be joined, using recombinant methods, by an artificial peptide linker that enables them to be made as a single protein chain. Such single chain antibodies include one or more antigen binding moieties. These antibody fragments are obtained using conventional techniques known to those of skill in the art, and the fragments are screened for utility in the same manner as are intact antibodies.

An antibody “that binds” an antigen of interest (e.g., hMPV F protein) is one that binds the antigen with sufficient affinity such that the antibody is useful as a therapeutic agent in targeting a cell or tissue expressing the antigen, and does not significantly cross-react with other proteins. In such embodiments, the extent of binding of the antibody to a “non-target” protein will be less than about 10% of the binding of the antibody, oligopeptide or other organic molecule to its particular target protein as determined, for example, by fluorescence activated cell sorting (FACS) analysis, enzyme-linked immunosorbent assay (ELISA), immunoprecipitation or radioimmunoprecipitation (RIA). With regard to the binding of an antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. The specific region of the antigen to which the antibody binds is typically referred to as an “epitope”. The term “epitope” broadly includes the site on an antigen which is specifically recognized by an antibody or T-cell receptor or otherwise interacts with a molecule. Generally epitopes are of active surface groupings of molecules such as amino acids or carbohydrate or sugar side chains and generally may have specific three-dimensional structural characteristics, as well as specific charge characteristics. As will be appreciated by one of skill in the art, practically anything to which an antibody can specifically bind could be an epitope.

An antibody “binds to” or is “specific to” or “specifically binds” (used interchangeably herein) to a target (e.g., human metapneumovirus or an hMPV protein) are terms well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. For example, an immunoglobulin that specifically or preferentially binds to thymocyte is an immunoglobulin that binds thymocytes with greater affinity, avidity, more readily, and/or with greater duration than it binds to other cells. An immunoglobulin that specifically binds to a first cell or substance may or may not specifically or preferentially bind to a second cell or substance. As such, “specific binding” does not necessarily require (although it can include) exclusive binding. Generally, but not necessarily, reference to binding means specific binding.

Throughout this disclosure, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. “Consisting essentially of” is meant to include any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The term “pharmaceutically acceptable excipients” refers to excipients biologically or pharmacologically compatible for in vivo use in animals or humans, and can mean excipients approved by a regulatory agency of the Federal or a state government or listed in the U.S. Pharmacopeia or other generally recognized pharmacopeia for use in animals, and more particularly in humans.

The term “adjuvant” refers to a pharmaceutically acceptable substance that enhances the immune response to an antigen when co-administered with the antigen or administered before, during, or after administration of the antigen to a subject.

“Effective amount”, in the context of treating a disease or condition, means the administration of an amount of an agent or composition to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the age, health and physical condition of the individual to be treated and whether symptoms of disease are apparent, the taxonomic group of individual to be treated, the formulation of the composition, the assessment of the medical situation, and other relevant factors. Optimal dosing schedules can be calculated from measurements of drug accumulation in the body of the subject. Optimum dosages may vary depending on the relative potency in an individual subject, and can generally be estimated based on EC50 values found to be effective in in vitro and in vivo animal models. Persons of ordinary skill can easily determine optimum dosages, dosing methodologies and repetition rates. It is expected that the amount will fall in a relatively broad range that can be determined through routine trials.

The term “therapeutically effective amount” or “effective dosage” as used herein refers to the dosage or concentration of a drug effective to treat a disease or condition. For example, with regard to the use of monoclonal antibodies or antigen-binding fragments to treat viral infection.

The term “immune response” refers to elicitation of activity of one or more immune cell types in the subject. Immune responses include, for example, T cell and B cell responses.

The term “humoral immune response” refers to an immune response that generates plasma or serum antibodies (e.g., IgG).

The term “administering” refers to providing a composition to a subject in a manner that permits the composition to have its intended effect. Administration for vaccination or post-exposure prophylaxis may be performed by intramuscular injection, intravenous injection, intraperitoneal injection, or any other suitable route.

The term “subject” refers to a human or non-human animal to which a composition may be administered for vaccination, treatment, or other purpose. In some embodiments, the non-human animal is a non-human primate, rabbit, hamster, gerbil, pig, cow, sheep, goat, guinea pig, rat, mouse, squirrel, wolf, fox, horse, zebra, giraffe, elephant, cat, dog, llama, or ferret.

The term “polynucleotide” refers to a polymeric form of nucleotides of more than about 100 nucleotides, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “identical” or percent “identity,” in the context of two or more polynucleotides or polypeptide sequences, refer to two or more sequences or subsequences that are the same or have a specified percentage of amino acid residues or nucleotides that are the same (i.e., share at least about 80% identity, for example, at least about 85%, 90%, 91%, 92%, 93%, 94%, 95%, 96%, 97%, 98%, 99% identity over a specified region to a reference sequence, when compared and aligned for maximum correspondence over a comparison window, or designated region as measured using one of the following sequence comparison algorithms or by manual alignment and visual inspection. This definition also refers to the compliment of a test sequence. In some embodiments, the identity exists over a region that is at least about 25 amino acids or nucleotides in length, for example, over a region that is 50, 100, 200, 300, 400 amino acids or nucleotides in length, or over the full-length of a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. In some embodiments, BLAST and BLAST 2.0 algorithms and the default parameters are used.

The terms “treatment”, “treating”, and the like, refer to one or more of relieving, alleviating, delaying, reducing, reversing, improving, or managing at least one symptom of a condition in a subject. The term “treating” may also mean one or more of arresting, delaying the onset (i.e., the period prior to clinical manifestation of the condition) or reducing the risk of developing or worsening a condition.

The articles “a” and “an” are used herein to refer to one or to more than one (i.e., to at least one) of the grammatical object of the article. By way of example, “an element” means one element or more than one element.

As used herein, “and/or” refers to and encompasses any and all possible combinations of one or more of the associated listed items, as well as the lack of combinations when interpreted in the alternative (or).

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation. Alternatively, “about” can mean plus or minus a range of up to 20%, up to 10%, or up to 5%.

All weight percentages (i.e., “% by weight” and “wt. %” and “w/w”) referenced herein, unless otherwise indicated, are measured relative to the total weight of the pharmaceutical composition.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof.

The following description includes information that may be useful in understanding the present invention. It is not an admission that any of the information provided herein is prior art or relevant to the presently claimed inventions, or that any publication specifically or implicitly referenced is prior art.

### Antigen-Binding Molecules

Provided are epitope-specific antigen-binding molecules specific to an hMPV Fusion (F) protein, wherein the antigen-binding molecule comprises a variable domain that contacts the hMPV F protein at
i.) residues 287, 293, 296, 364, 376, 417 and 419;
ii.) residues 144, 160, 163, 188, 194 and 199; or
iii.) residues 44, 45, 49, 150, 156, 160, 229, 232 and 236 of the hMPV F protein amino acid sequence set forth in SEQ ID NO: 1.

hMPV033 F protein is used as a reference sequence:

(SEQ ID NO: 1)
MSWKVVIIFSLLITPQHGLKESYLEESCSTITEGYLSVLRTGWYTNVFTLEVG
DVENLTCADGPSLIKTELDLTKSALRELRTVSADQLAREEQIEGGGGGGFVL
GAIALGVATAAAVTAGVAIAKCIRLESEVTAIKNALKKTNEAVSTLGCGVR
VLATAVRELKDFVSKNLTRAINKNKCDIPDLKMAVSFSQFNRRFLNVVRQF
SDNAGITPAISKDLMTDAELARAISNMPTSAGQIKLMLENRAMVRRKGFGI
LIGVYGSSVIYMVOLPIFGVIDTPCWIVKAAPSCSEKKGNYACLLREDQGW
YCQNAGSTVYYPNEKDCETRGDHVFCDTAAGINVAEQSKECNINISTTNYP
CKVSCGRNPISMVALSPLGALVACYKGVSCSIGSNRVGIIKQLNKGCSYITN
QDADTVTIDNTVYQLSKVEGEQHVIKGRPVSSSFDPVKFPEDQFNVALDQC
FESIENSQAGSGGSGSGSGGSEKAAKAEEAARKMEELFKKHKIVAVLRANS
VEEAIEKAVAVFAGGVHLIEITFTVPDADTVIKALSVLKEKGAIIGAGTVTSV
EQARKAVESGAEFIVSPHLDEEISQFAKEKGVFYMPGVMTPTELVKAMKLG
HTILKLFPGEVVGPQFVKAMKGPFPNVKFVPTGGVNLDNVAEWFKAGVLA
VGVGSALVKGTPDEVREKAKAFVEKIRGATELE

In some embodiments, the epitope-specific antigen-binding molecule specific to an hMPV Fusion (F) protein is an antibody. In some embodiments, the epitope-specific antigen-binding molecule specific to an hMPV Fusion (F) protein is a neutralizing antibody.

In some embodiments, the epitope-specific antigen-binding molecule specific to an hMPV Fusion (F) protein comprises a variable domain that contacts the hMPV F protein at residues 287, 293, 296, 364, 376, 417 and 419 of the hMPV F protein amino acid sequence set forth in SEQ ID NO: 1 and may be referred to as the “17D10” antigen-binding molecule.

In some embodiments, the epitope-specific antigen-binding molecule specific to an hMPV Fusion (F) protein comprises a variable domain that contacts the hMPV F protein at residues 144, 160, 163, 188, 194 and 199 of the hMPV F protein amino acid sequence set forth in SEQ ID NO: 1 and may be referred to as the “13E10” antigen-binding molecule.

In some embodiments, the epitope-specific antigen-binding molecule specific to an hMPV Fusion (F) protein comprises a variable domain that contacts the hMPV F protein at residues 44, 45, 49, 150, 156, 160, 229, 232 and 236 of the hMPV F protein amino acid sequence set forth in SEQ ID NO: 1 and may be referred to as the “42C2” antigen-binding molecule.

Illustrative antigen-binding molecule heavy and light chain sequences are provided in Table 1.

TABLE 1

| Name | Domain | Amino Acid Sequence |
|---|---|---|
| 17D10 | heavy | QVQLQQPGAELVKPGASVKMSCKASGYTFTSYWITWVKQRPGQ |
| hIgG1 | chain | GLEWIGDIYPGSGSITYNEKFKTKATLTVDTSSSTAYMQLNSLTSE |
| heavy | | DSAVYYCAGLLRLTFDVWGTGTTVTVSSASTKGPSVFPLAPSSKS |
| chain | | TSGGTAALGCLVKDYFPEPVTVSWNSGALTSGVHTFPAVLQSSG |
| | | LYSLSSVVTVPSSSLGTQTYICNVNHKPSNTKVDKKVEPKSCDKT |
| | | HTCPPCPAPELLGGPSVFLFPPKPKDTLMISRTPEVTCVVVDVSHE |
| | | DPEVKFNWYVDGVEVHNAKTKPREEQYNSTYRVVSVLTVLHQD |
| | | WLNGKEYKCKVSNKALPAPIEKTISKAKGQPREPQVYTLPPSRDE |
| | | LTKNQVSLTCLVKGFYPSDIAVEWESNGQPENNYKTTPPVLDSD |
| | | GSFFLYSKLTVDKSRWQQGNVFSCSVMHEALHNHYTQKSLSLSP |
| | | GK (SEQ ID NO: 2) |
| 17D10 | light | DIQLTQTTSSLSASLGDRVTISCRASQDISNYLNWYQQKPDGTVK |
| hIgG1 | chain | LLISYTSGLHSGVPSRFSGSGSGTDYSLTISNLEQEDIATYFCQQGN |
| light | | TLPWTFGGGTKLEIKRTVAAPSVFIFPPSDEQLKSGTASVVCLLNN |
| chain | | FYPREAKVQWKVDNALQSGNSQESVTEQDSKDSTYSLSSTLTLS |
| | | KADYEKHKVYACEVTHQGLSSPVTKSFNRGEC (SEQ ID NO: 6) |
| 13E10 | heavy | EVKLVESGGGLVQPGGSLSLSCAASGFTFTDYYMNWVRQPPGK |
| hIgG1 | chain | ALEWLGFIRNKDNGYTTEYSASVKGRFTISRDNSQSILYLQMNAL |
| heavy | | RAEDSATYYCTRYYFGYDGDYFDYWGQGTTLTVSSASTKGPSVF |
| chain | | PLAPSSKSTSGGTAALGCLVKDYFPEPVTVSWNSGALTSGVHTFP |
| | | AVLQSSGLYSLSSVVTVPSSSLGTQTYICNVNHKPSNTKVDKKVE |
| | | PKSCDKTHTCPPCPAPELLGGPSVFLFPPKPKDTLMISRTPEVTCV |
| | | VVDVSHEDPEVKFNWYVDGVEVHNAKTKPREEQYNSTYRVVSV |
| | | LTVLHQDWLNGKEYKCKVSNKALPAPIEKTISKAKGQPREPQVY |
| | | TLPPSRDELTKNQVSLTCLVKGFYPSDIAVEWESNGQPENNYKTT |
| | | PPVLDSDGSFFLYSKLTVDKSRWQQGNVFSCSVMHEALHNHYTQ |
| | | KSLSLSPGK (SEQ ID NO: 10) |
| 13E10 | light | EIVLTQSPTTMAASPGEKITITCSASSSISSNYLHWYQQKAGFSPKL |
| hIgG1 | chain | LIYRTSNLASGVPARFSGSGSGTSYSLTIGTMEAEDVATYYCQQG |
| light | | SSLPRTFGGGTKLEIKRTVAAPSVFIFPPSDEQLKSGTASVVCLLN |
| chain | | NFYPREAKVQWKVDNALQSGNSQESVTEQDSKDSTYSLSSTLTL |
| | | SKADYEKHKVYACEVTHQGLSSPVTKSFNRGEC (SEQ ID NO: 14) |
| 42C2 | heavy | QVTLKESGPGILQPSQTLSLTCSFSGFSLSTFGMGVVWTRQPSGK |
| hIgG1 | chain | GLEWLAHIWWDDDNYYNPALKSRLTISKDTSKNQVFLKIANADT |
| heavy | | ADTATYYCARIVKVLEQYFDVWGTGTTVTVSSASTKGPSVFPLA |
| chain | | PSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKS |
| | | CDKTHTCPPCPAPELLGGPSVFLFPPKPKDTLMISRTPEVTCVVVD |
| | | VSHEDPEVKFNWYVDGVEVHNAKTKPREEQYNSTYRVVSVLTV |
| | | LHQDWLNGKEYKCKVSNKALPAPIEKTISKAKGQPREPQVYTLP |
| | | PSRDELTKNQVSLTCLVKGFYPSDIAVEWESNGQPENNYKTTPPV |
| | | LDSDGSFFLYSKLTVDKSRWQQGNVFSCSVMHEALHNHYTQKS |
| | | LSLSPGK (SEQ ID NO: 18) |
| 42C2 | light | DIMMTQSHKFMSTSVGDRVIFTCKASQDVGTAVAWYQQKSGQS |
| hIgG1 | chain | PKLLIYWASTRHTGVPDRFTGSGSGTDFTLTISNVQSEDLADYFC |
| light | | QQYTSYPLTFGAGTKLELKRTVAAPSVFIFPPSDEQLKSGTASVV |
| chain | | CLLNNFYPREAKVQWKVDNALQSGNSQESVTEQDSKDSTYSLSS |
| | | TLTLSKADYEKHKVYACEVTHQGLSSPVTKSFNRGEC (SEQ ID |
| | | NO: 22) |

Illustrative antigen-binding molecule variable domain sequences are provided in Table 2.

TABLE 2

| | | | Contact residues on the |
|---|---|---|---|
| | | | hMPV F protein amino |
| | | | acid sequence set forth |
| Name | Domain | Amino Acid Sequence | in SEQ ID NO: 1 |
| 17D10 | VH | QVQLQQPGAELVKPGASVKMSC | 287, 293, 296, 364, 376, |
| hIgG1 VH | | KASGYTFTSYWITWVKQRPGQG | 417, 419 |
| | | LEWIGDIYPGSGSITYNEKFKTKA | |
| | | TLTVDTSSSTAYMQLNSLTSEDS | |
| | | AVYYCAGLLRLTFDVWGTGTTV | |
| | | TVSS (SEQ ID NO: 50) | |
| 17D10 | VL | DIQLTQTTSSLSASLGDRVTISCR | 287, 293, 296, 364, 376, |
| hIgG1 VL | | ASQDISNYLNWYQQKPDGTVKL | 417, 419 |
| | | LISYTSGLHSGVPSRFSGSGSGTD | |
| | | YSLTISNLEQEDIATYFCQQGNTL | |
| | | PWTFGGGTKLEIK (SEQ ID NO: | |
| | | 51) | |
| 13E10 | VH | EVKLVESGGGLVQPGGSLSLSCA | 144, 160, 163, 188, 194, |
| hIgG1 VH | | ASGFTFTDYYMNWVRQPPGKAL | 199 |
| | | EWLGFIRNKDNGYTTEYSASVK | |
| | | GRFTISRDNSQSILYLQMNALRA | |
| | | EDSATYYCTRYYFGYDGDYFDYWG | |
| | | QGTTLTVSS (SEQ ID NO: 52) | |
| 13E10 | VL | EIVLTQSPTTMAASPGEKITITCSA | 144, 160, 163, 188, 194, |
| hIgG1 VL | | SSSISSNYLHWYQQKAGFSPKLLI | 199 |
| | | YRTSNLASGVPARFSGSGSGTSYS | |
| | | LTIGTMEAEDVATYYCQQGSSLPRT | |
| | | FGGGTKLEIK (SEQ ID NO: 53) | |
| 42C2 | VH | QVTLKESGPGILQPSQTLSLTCSF | 44, 45, 49, 150, 156, |
| hIgG1 VH | | SGFSLSTFGMGVVWTRQPSGKG | 160, 229, 232, 236 |
| | | LEWLAHIWWDDDNYYNPALKS | |
| | | RLTISKDTSKNQVFLKIANADTA | |
| | | DTATYYCARIVKVLEQYFDVWG | |
| | | TGTTVTVSS (SEQ ID NO: 54) | |
| 42C2 | VL | DIMMTQSHKFMSTSVGDRVIFTC | 44, 45, 49, 150, 156, |
| hIgG1 VL | | KASQDVGTAVAWYQQKSGQSP | 160, 229, 232, 236 |
| | | KLLIYWASTRHTGVPDRFTGSGS | |
| | | GTDFTLTISNVQSEDLADYFCQQ | |
| | | YTSYPLTFGAGTKLELK (SEQ ID | |
| | | NO: 55) | |

Illustrative antigen-binding molecule variable domain CDR sequences are provided in Table 3.

TABLE 3

| Name | Domain | Amino Acid Sequence |
|---|---|---|
| 17D10 hIgG1 VH | H-CDR-1 | GYTFTSY (SEQ ID NO: 3) |
| 17D10 hIgG1 VH | H-CDR-2 | YPGSGS (SEQ ID NO: 4) |
| 17D10 hIgG1 VH | H-CDR-3 | LLRLTFDV (SEQ ID NO: 5) |
| 17D10 hIgG1 VL | L-CDR-1 | RASQDISNYLN (SEQ ID NO: 7) |
| 17D10 hIgG1 VL | L-CDR-2 | YTSGLHS (SEQ ID NO: 8) |
| 17D10 hIgG1 VL | L-CDR-3 | QQGNTLPWT (SEQ ID NO: 9) |
| 13E10 hIgG1 VH | H-CDR-1 | GFTFTDY (SEQ ID NO: 11) |
| 13E10 hIgG1 VH | H-CDR-2 | RNKDNGYT (SEQ ID NO: 12) |
| 13E10 hIgG1 VH | H-CDR-3 | YYFGYDGDYFDY (SEQ ID NO: 13) |
| 13E10 hIgG1 VL | L-CDR-1 | SASSSISSNYLH (SEQ ID NO: 15) |
| 13E10 hIgG1 VL | L-CDR-2 | RTSNLAS (SEQ ID NO: 16) |
| 13E10 hIgG1 VL | L-CDR-3 | QQGSSLPRT (SEQ ID NO: 17) |
| 42C2 hIgG1 VH | H-CDR-1 | GFSLSTFGM (SEQ ID NO: 19) |
| 42C2 hIgG1 VH | H-CDR-2 | WWDDD (SEQ ID NO: 20) |
| 42C2 hIgG1 VH | H-CDR-3 | IVKVLEQYFDV (SEQ ID NO: 21) |
| 42C2 hIgG1 VL | L-CDR-1 | KASQDVGTAVA (SEQ ID NO: 23) |
| 42C2 hIgG1 VL | L-CDR-2 | WASTRHT (SEQ ID NO: 24) |
| 42C2 hIgG1 VL | L-CDR-3 | QQYTSYPLT (SEQ ID NO: 25) |

Illustrative antigen-binding molecule variable domain epitope contact residues are provided in Table 4.

TABLE 4

| Antigen-Binding | |
|---|---|
| Molecule Name | Variable Domain Epitope Contact Residues |
| 17D10 | 287, 293, 296, 364, 376, 417, and 419 of the |
| | hMPV F protein amino acid sequence set |
| | forth in SEQ ID NO: 1 |
| 13E10 | 144, 160, 163, 188, 194, and 199 of the |
| | hMPV F protein amino acid sequence set |
| | forth in SEQ ID NO: 1 |
| 42C2 | 44, 45, 49, 150, 156, 160, 229, 232, and 236 |
| | of the hMPV F protein amino acid sequence |
| | set forth in SEQ ID NO: 1 |

### Epitope I—17D10

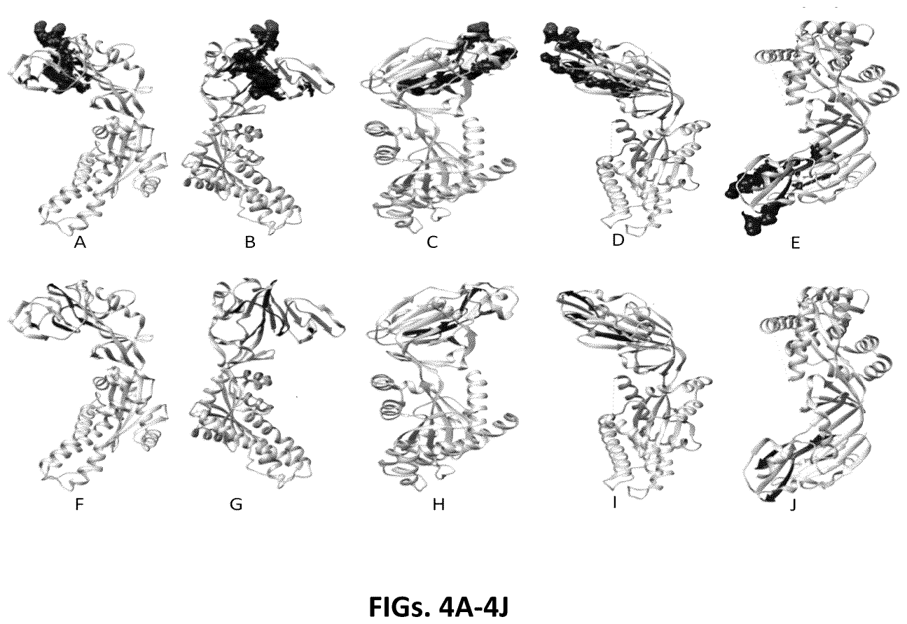

The present disclosure contemplates any hMPV F protein antigen-binding molecule that binds to hMPV F protein, such as human hMPV F protein in both the pre-fusion and post-fusion hMPV F protein conformations. In some embodiments, the hMPV F protein antigen-binding molecule is a 17D10 antibody that binds an epitope comprised of at least a portion of the region from amino acid 287 to amino acid 419 of human hMPV F protein. As shown in FIGS. 4A-4J, these amino acid residues form a non-linear epitope on the hMPV F protein. The anti-hMPV F protein antigen-binding molecule of the present disclosure that binds the 17D10 epitope binds hMPV F protein in the pre-fusion and post-fusion confirmations.

In some embodiments, the epitope-specific antigen-binding molecule specific to an hMPV Fusion (F) protein is an antibody. In some embodiments, the epitope-specific antigen-binding molecule specific to an hMPV Fusion (F) protein is a neutralizing antibody.

In some embodiments, the epitope-specific antigen-binding molecule specific to an hMPV Fusion (F) protein comprises a variable domain that contacts the hMPV F protein at residues 287, 293, 296, 364, 376, 417 and 419 of the hMPV F protein amino acid sequence set forth in SEQ ID NO: 1 and may be referred to as the “17D10” antigen-binding molecule.

In some embodiments, an antigen-binding molecule comprises a variable domain that contacts the hMPV F protein at any 1, any 2, any 3, any 4, any 5, any 6, or any 7 of residues 287, 293, 296, 364, 376, 417 and 419 of the hMPV F protein amino acid sequence set forth in SEQ ID NO: 1.

In some embodiments, an antigen-binding molecule “17D10” comprises a heavy chain and a light chain, wherein the heavy chain comprises an H-CDR1, an H-CDR2, and an H-CDR3 wherein: H-CDR1 comprises the sequence GYTFTSY (SEQ ID NO: 3); H-CDR2 comprises the sequence YPGSGS (SEQ ID NO: 4); and H-CDR3 comprises the sequence LLRLTFDV (SEQ ID NO: 5).

In some embodiments, an antigen-binding molecule comprises a light chain comprising an L-CDR1, an L-CDR2, and an L-CDR3, wherein: L-CDR1 comprises the sequence RASQDISNYLN (SEQ ID NO: 7); L-CDR2 comprises the sequence YTSGLHS (SEQ ID NO: 8); and L-CDR3 comprises the sequence QQGNTLPWT (SEQ ID NO: 9).

In some embodiments, an antigen-binding molecule “17D10” specific to an hMPV F protein comprises a heavy chain and a light chain, wherein the heavy chain comprises an H-CDR1, an H-CDR2, and an H-CDR3 wherein: H-CDR1 comprises the sequence GYTFTSY (SEQ ID NO: 3); H-CDR2 comprises the sequence YPGSGS (SEQ ID NO: 4); and H-CDR3 comprises the sequence LLRLTFDV (SEQ ID NO: 5); and wherein the light chain comprises an L-CDR1, an L-CDR2, and an L-CDR3, wherein: L-CDR1 comprises the sequence RASQDISNYLN (SEQ ID NO: 7); L-CDR2 comprises the sequence YTSGLHS (SEQ ID NO: 8); and L-CDR3 comprises the sequence QQGNTLPWT (SEQ ID NO: 9).

In some embodiments, the heavy chain comprises a sequence having at least 70%, at least 80%, at least 90%, at least 95%, at least 97%, at least 98%, at least 99%, or 100% sequence identity to SEQ ID NO: 2.

In some embodiments, the light chain comprises a sequence having at least 70%, at least 80%, at least 90%, at least 95%, at least 97%, at least 98%, at least 99%, or 100% sequence identity SEQ ID NO: 6.

In some embodiments, the heavy chain comprises a sequence having at least 70%, at least 80%, at least 90%, at least 95%, at least 97%, at least 98%, at least 99%, or 100% sequence identity to SEQ ID NO: 2, and the light chain comprises a sequence having at least 70%, at least 80%, at least 90%, at least 95%, at least 97%, at least 98%, at least 99%, or 100% sequence identity to SEQ ID NO: 6.

In some embodiments, the antigen-binding molecule comprises a variable heavy (VH) chain domain wherein the VH domain comprises a sequence having at least 70%, at least 80%, at least 90%, at least 95%, at least 97%, at least 98%, at least 99%, or 100% sequence identity to SEQ ID NO: 50.

In some embodiments, the antigen-binding molecule comprises a variable light (VL) chain domain wherein the VL domain comprises a sequence having at least 70%, at least 80%, at least 90%, at least 95%, at least 97%, at least 98%, at least 99%, or 100% sequence identity to SEQ ID NO: 51.

In some embodiments, the antigen-binding molecule comprises a variable heavy (VH) chain domain wherein the VH domain comprises a sequence having at least 70%, at least 80%, at least 90%, at least 95%, at least 97%, at least 98%, at least 99%, or 100% sequence identity to SEQ ID NO: 50, and a variable light (VL) chain domain wherein the VL domain comprises a sequence having at least 70%, at least 80%, at least 90%, at least 95%, at least 97%, at least 98%, at least 99%, or 100% sequence identity to SEQ ID NO: 51.

### Epitope II—13E10

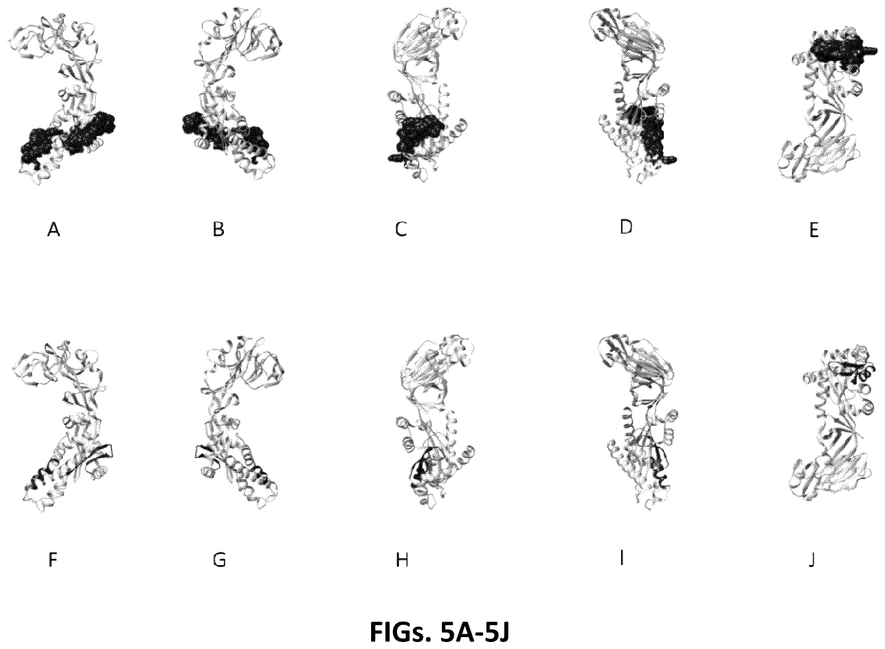

The present disclosure contemplates any hMPV F protein antigen-binding molecule that binds to hMPV F protein, specifically in the pre-fusion hMPV F protein conformation. In some embodiments, the hMPV F protein antigen-binding molecule is a 13E10 antibody that binds an epitope comprised in at least a portion of the region from amino acid 144 to amino acid 199 of hMPV F protein. As shown in FIGS. 5A-5J, these amino acid residues form a non-linear epitope on the hMPV F protein.

The anti-hMPV F protein antigen-binding molecule of the present disclosure that binds the 13E10 epitope only binds hMPV F protein in the pre-fusion confirmation.

In some embodiments, the epitope-specific antigen-binding molecule specific to an hMPV Fusion (F) protein is an antibody. In some embodiments, the epitope-specific antigen-binding molecule specific to an hMPV Fusion (F) protein is a neutralizing antibody.

In some embodiments, the epitope-specific antigen-binding molecule specific to an hMPV Fusion (F) protein comprises a variable domain that contacts the hMPV F protein at residues 144, 160, 163, 188, 194 and 199 of the hMPV F protein amino acid sequence set forth in SEQ ID NO: 1 and may be referred to as the “13E10” antigen-binding molecule.

In some embodiments, the antigen-binding molecule comprises a variable domain that contacts the hMPV F protein at any 1, any 2, any 3, any 4, any 5, or any 6 of residues 144, 160, 163, 188, 194 and 199 of the hMPV F protein amino acid sequence set forth in SEQ ID NO: 1.

In some embodiments, the antigen-binding molecule “13E10” comprises a heavy chain and a light chain, wherein the heavy chain comprises an H-CDR1, an H-CDR2, and an H-CDR3 wherein: H-CDR1 comprises the sequence GFTFTDY (SEQ ID NO: 11); H-CDR2 comprises the sequence RNKDNGYT (SEQ ID NO: 12); and H-CDR3 comprises the sequence YYFGYDGDYFDY (SEQ ID NO: 13); and wherein the light chain comprises an L-CDR1, an L-CDR2, and an L-CDR3, wherein: L-CDR1 comprises the sequence SASSSISSNYLH (SEQ ID NO: 15); L-CDR2 comprises the sequence RTSNLAS (SEQ ID NO: 16); and L-CDR3 comprises the sequence QQGSSLPRT (SEQ ID NO: 17).

In some embodiments, the heavy chain comprises a sequence having at least 70%, at least 80%, at least 90%, at least 95%, at least 97%, at least 98%, at least 99%, or 100% sequence identity to SEQ ID NO: 10.

In some embodiments, the light chain comprises a sequence having at least 70%, at least 80%, at least 90%, at least 95%, at least 97%, at least 98%, at least 99%, or 100% sequence identity SEQ ID NO: 14.

In some embodiments, the heavy chain comprises a sequence having at least 70%, at least 80%, at least 90%, at least 95%, at least 97%, at least 98%, at least 99%, or 100% sequence identity to SEQ ID NO: 10, and the light chain comprises a sequence having at least 70%, at least 80%, at least 90%, at least 95%, at least 97%, at least 98%, at least 99%, or 100% sequence identity to SEQ ID NO: 14.

In some embodiments, the antigen-binding molecule comprises a variable heavy (VH) chain domain wherein the VH domain comprises a sequence having at least 70%, at least 80%, at least 90%, at least 95%, at least 97%, at least 98%, at least 99%, or 100% sequence identity to SEQ ID NO: 52.

In some embodiments, the antigen-binding molecule comprises a variable light (VL) chain domain wherein the VL domain comprises a sequence having at least 70%, at least 80%, at least 90%, at least 95%, at least 97%, at least 98%, at least 99%, or 100% sequence identity to SEQ ID NO: 53.

In some embodiments, the antigen-binding molecule comprises a variable heavy (VH) chain domain wherein the VH domain comprises a sequence having at least 70%, at least 80%, at least 90%, at least 95%, at least 97%, at least 98%, at least 99%, or 100% sequence identity to SEQ ID NO: 52, and a variable light (VL) chain domain wherein the VL domain comprises a sequence having at least 70%, at least 80%, at least 90%, at least 95%, at least 97%, at least 98%, at least 99%, or 100% sequence identity to SEQ ID NO: 53.

### Epitope III—42C2

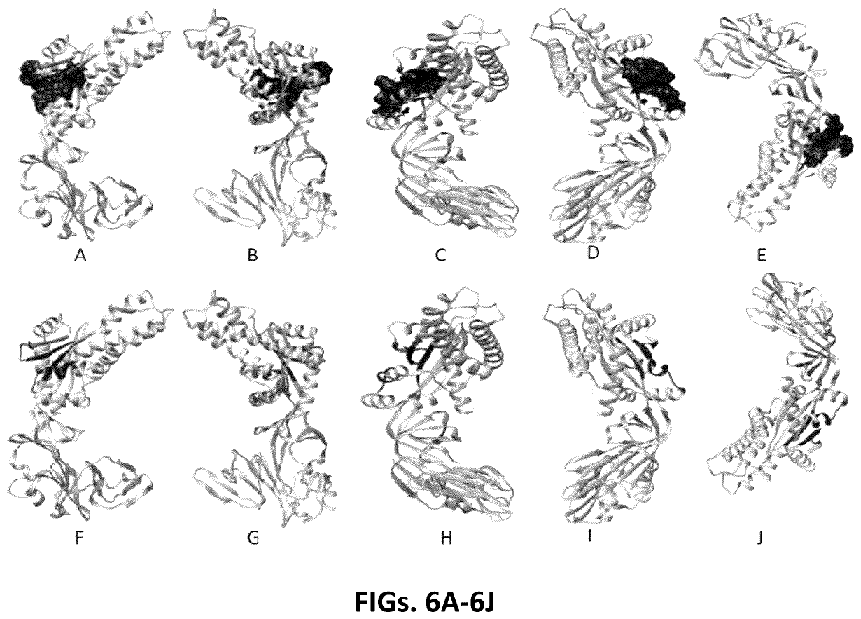

The present disclosure contemplates any hMPV F protein antigen-binding molecule that binds to hMPV F protein in both the pre-fusion and post-fusion hMPV F protein conformations. In some embodiments, the hMPV F protein antigen-binding molecule is a 42C2 antibody that binds an epitope comprised in at least a portion of the region from amino acid 44 to amino acid 236 of hMPV F protein. As shown in FIGS. 6A-6J, these amino acid residues form a non-linear epitope on the hMPV F protein.

The anti-hMPV F protein antibody of the present disclosure that binds the 42C2 epitope binds hMPV F protein in the pre-fusion and post-fusion confirmations.

In some embodiments, the epitope-specific antigen-binding molecule specific to an hMPV Fusion (F) protein is an antibody. In some embodiments, the epitope-specific antigen-binding molecule specific to an hMPV Fusion (F) protein is a neutralizing antibody.

In some embodiments, the epitope-specific antigen-binding molecule specific to an hMPV Fusion (F) protein comprises a variable domain that contacts the hMPV F protein at residues 44, 45, 49, 150, 156, 160, 229, 232 and 236 of the hMPV F protein amino acid sequence set forth in SEQ ID NO: 1 and may be referred to as the “42C2” antigen-binding molecule.

In some embodiments, the antigen-binding molecule comprises a variable domain that contacts the hMPV F protein at any 1, any 2, any 3, any 4, any 5, any 6, any 7, any 8, or any 9 of residues 44, 45, 49, 150, 156, 160, 229, 232 and 236 of the hMPV F protein amino acid sequence set forth in SEQ ID NO: 1.

In some embodiments, the antigen-binding molecule “42C2” comprises a heavy chain and a light chain, wherein the heavy chain comprises an H-CDR1, an H-CDR2, and an H-CDR3 wherein: H-CDR1 comprises the sequence GFSLSTFGM (SEQ ID NO: 19); H-CDR2 comprises the sequence WWDDD (SEQ ID NO: 20); and H-CDR3 comprises the sequence IVKVLEQYFDV (SEQ ID NO: 21); and wherein the light chain comprises an L-CDR1, an L-CDR2, and an L-CDR3, wherein: L-CDR1 comprises the sequence KASQDVGTAVA (SEQ ID NO: 23); L-CDR2 comprises the sequence WASTRHT (SEQ ID NO: 24); and L-CDR3 comprises the sequence QQYTSYPLT (SEQ ID NO: 25).

In some embodiments, the heavy chain comprises a sequence having at least 70%, at least 80%, at least 90%, at least 95%, at least 97%, at least 98%, at least 99%, or 100% sequence identity to SEQ ID NO: 18.

In some embodiments, the light chain comprises a sequence having at least 70%, at least 80%, at least 90%, at least 95%, at least 97%, at least 98%, at least 99%, or 100% sequence identity SEQ ID NO: 22.

In some embodiments, the heavy chain comprises a sequence having at least 70%, at least 80%, at least 90%, at least 95%, at least 97%, at least 98%, at least 99%, or 100% sequence identity to SEQ ID NO: 18, and the light chain comprises a sequence having at least 70%, at least 80%, at least 90%, at least 95%, at least 97%, at least 98%, at least 99%, or 100% sequence identity to SEQ ID NO: 22.

In some embodiments, the antigen-binding molecule comprises a variable heavy (VH) chain domain wherein the VH domain comprises a sequence having at least 70%, at least 80%, at least 90%, at least 95%, at least 97%, at least 98%, at least 99%, or 100% sequence identity to SEQ ID NO: 54.

In some embodiments, the antigen-binding molecule comprises a variable light (VL) chain domain wherein the VL domain comprises a sequence having at least 70%, at least 80%, at least 90%, at least 95%, at least 97%, at least 98%, at least 99%, or 100% sequence identity to SEQ ID NO: 55.

In some embodiments, the antigen-binding molecule comprises a variable heavy (VH) chain domain wherein the VH domain comprises a sequence having at least 70%, at least 80%, at least 90%, at least 95%, at least 97%, at least 98%, at least 99%, or 100% sequence identity to SEQ ID NO: 54, and a variable light (VL) chain domain wherein the VL domain comprises a sequence having at least 70%, at least 80%, at least 90%, at least 95%, at least 97%, at least 98%, at least 99%, or 100% sequence identity to SEQ ID NO: 55.

In some embodiments, the antigen-binding molecule is an immunoglobulin molecule.

In some embodiments, the immunoglobulin is an IgG1, IgG2, IgG3, or IgG4 molecule.

In some embodiments, the immunoglobulin is a humanized antibody.

In some embodiments, the immunoglobulin is a neutralizing antibody.

The hMPV F Protein

Human metapneumovirus (hMPV) is a negative-sense enveloped virus of the Pneumoviridae family and was discovered in 2001 but has been circulating for at least a half century before its discovery. The hMPV fusion (F) protein is one of three surface glycoproteins encoded by the viral genome. As a class I fusion, hMPV F is first translated as a single polypeptide precursor (F0). Initially nonfunctional, a proteolytic cleavage event is necessary to form the F1 and F2 subunits which are covalently linked by disulfide bonds. The new N-terminus of the F2 polypeptide contains a hydrophobic sequence which gets inserted into the host-cell membrane during the process that eventually fuses the viral and host-cell membranes. At some point, either in transit or at the membrane surface, the F protein associates with itself to form a metastable trimer in what is termed to be the pre-fusion conformation. The hMPV fusion protein is cleaved extracellularly by a trypsin-like protease. An unknown triggering event occurs that influences the F protein to undergo a dramatic conformational change, extending the fusion peptide into the host-cell membrane, before collapsing back onto itself to form a six-helix bundle in what is termed the post-fusion conformation. The energy differential between the elongated intermediate and the post-fusion conformation provides the energy necessary for membrane fusion.

The present disclosure provides methods, uses, and compositions that include an hMPV F protein antigen-binding molecule for treating hMPV infection in a subject. The present disclosure also provides methods, uses, and compositions that include an hMPV F protein antigen-binding molecule for treating hMPV.

The hMPV033 antigen (SEQ ID NO: 1) comprises the ectodomain (1-472 AA) of the UT-A CL-28 mutant. The CL-28 mutant strain was constructed in the A strain sequence with a 6 amino acid Gly linker replacing the F1/F2 cleavage site. The mutant also contains the following mutations: T127C, N153C, A185P, V231I, L219K, G294E, T365C, and V463C. In some embodiments, the mutant contains 368N. In some embodiments, the mutant contains 368H. In some embodiments, the mutant contains T127C, N153C, A185P, V231I, L219K, G294E, T365C, V463C, and H368N. The 127C-153C amino acid bond and the 365C-463C amino acid bond form intraprotomer disulfide bonds.

The hMPV F protein antigen-binding molecule may be a full-length immunoglobulin antibody or an antigen-binding fragment of an intact antibody, representative examples of which include a Fab fragment, a F(ab′)2 fragment, an Fd fragment consisting of the VH and CH1 domains, an Fv fragment consisting of the VL and VH domains of a single arm of an antibody, a single domain antibody (dAb) fragment (Ward et al., 1989. Nature 341:544-546), which consists of a VH domain; and an isolated CDR. In some embodiments, the hMPV F protein antigen-binding molecule is a chimeric, humanized or human antibody.

In some embodiments, the hMPV F protein antigen-binding molecule is a humanized antibody. Techniques for producing humanized monoclonal antibodies (mAbs) are well known in the art (see, for example, Jones et al., 1986. Nature 321: 522-525; Riechmann et al. 1988. Nature 332:323-329; Verhoeyen et al., 1988. Science 239: 1534-1536; Carter et al., 1992. Proc. Natl. Acad. Sci. USA 89: 4285-4289; Sandhu, J S., 1992. Crit. Rev. Biotech. 12: 437-462, and Singer et al., 1993. J. Immunol. 150: 2844-2857). A chimeric or murine monoclonal antibody may be humanized by transferring the mouse CDRs from the heavy and light variable chains of the mouse immunoglobulin into the corresponding variable domains of a human antibody. The mouse framework regions (FR) in the chimeric monoclonal antibody are also replaced with human FR sequences. As simply transferring mouse CDRs into human FRs often results in a reduction or even loss of antibody affinity, additional modification might be required in order to restore the original affinity of the murine antibody. This can be accomplished by the replacement of one or more human residues in the FR regions with their murine counterparts to obtain an antibody that possesses good binding affinity to its epitope. See, for example, Tempest et al. (1991. Biotechnology 9:266-271) and Verhoeyen et al. (1988 supra). Generally, those human FR amino acid residues that differ from their murine counterparts and are located close to or touching one or more CDR amino acid residues would be candidates for substitution.

### Polynucleotide

Provided are polynucleotides encoding an antigen-binding molecule of the present disclosure.

In an aspect, the present disclosure provides isolated polynucleotides encoding an antigen-binding molecule of the present disclosure. The isolated polynucleotide sequence may comprise RNA or DNA. As used herein, “isolated nucleic acids” are those that have been removed from their normal surrounding polynucleotide sequences in the genome or in cDNA sequences.

In a further aspect, the present disclosure provides recombinant expression vectors comprising the isolated polynucleotide of any embodiment or combination of embodiments of the disclosure operatively linked to a suitable control sequence. “Recombinant expression vector” includes vectors that operatively link a polynucleotide coding region or gene to any control sequences capable of effecting expression of the gene product. “Control sequences” operably linked to the polynucleotide sequences of the disclosure are polynucleotide sequences capable of effecting the expression of the polynucleotide molecules. The control sequences need not be contiguous with the polynucleotide sequences, so long as they function to direct the expression thereof. Thus, for example, intervening untranslated yet transcribed sequences can be present between a promoter sequence and the polynucleotide sequences, and the promoter sequence can still be considered “operably linked” to the coding sequence. Other such control sequences include, but are not limited to, polyadenylation signals, termination signals, and ribosome binding sites. Such recombinant expression vectors can be of any type known in the art, including but not limited to plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid responsive).

### Pharmaceutical Compositions

Provided is a pharmaceutical composition comprising an antigen-binding molecule of the present disclosure, and a pharmaceutically acceptable carrier, diluent, or excipient. Carrier(s) are “pharmaceutically acceptable” in the sense of being compatible with the other ingredients of the composition and not deleterious to the recipient thereof (e.g., subject). Suitable carriers typically include physiological saline or ethanol polyols such as glycerol or propylene glycol.

The antigen-binding molecule may be formulated as neutral or salt forms. Pharmaceutically acceptable salts include the acid addition salts (formed with free amino groups) and which are formed with inorganic acids such as hydrochloric or phosphoric acids, or such organic acids such as acetic, oxalic, tartaric and maleic. Salts formed with the free carboxyl groups may also be derived from inorganic bases such as sodium, potassium, ammonium, calcium, or ferric hydroxides, and organic bases as isopropylamine, trimethylamine, 2-ethylamino ethanol, histidine and procaine.

The compositions may be suitably formulated for systemic administration, including intravenous, intramuscular, subcutaneous, or intraperitoneal administration and conveniently comprise sterile aqueous solutions of the antigen-binding molecule, which are preferably isotonic with the blood of the recipient. Such formulations are typically prepared by dissolving the solid active ingredient in water containing physiologically compatible substances such as sodium chloride, glycine, and the like, and having a buffered pH compatible with physiological conditions to produce an aqueous solution, and rendering said solution sterile. These may be prepared in unit or multi-dose containers, for example, sealed ampoules or vials.

The compositions may incorporate a stabilizer, such as for example polyethylene glycol, proteins, saccharides (for example trehalose), amino acids, inorganic acids and admixtures thereof. Stabilizers are used in aqueous solutions at the appropriate concentration and pH. The pH of the aqueous solution is adjusted to be within the range of 5.0-9.0, preferably within the range of 6-8. In formulating the antigen-binding molecule, an anti-adsorption agent may be used. Other suitable excipients may typically include an antioxidant such as ascorbic acid.

In certain embodiments, the compositions disclosed herein may be used as a medicament, e.g., for use in treating or preventing infection in a subject in need thereof, such as a mammal.

In certain embodiments, the compositions disclosed herein may be used in the manufacture of a medicament for treating or preventing infection in a subject in need thereof, such as a mammal.

### Therapeutic Applications

Provided is a method of treating or preventing an hMPV infection in a subject in need thereof, wherein the method comprises administering to the subject an effective amount of an antigen-binding molecule according to the present disclosure.

In some embodiments, methods of the present disclosure comprise therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. A subject is successfully “treated” for an infection if, after receiving an effective amount of an antigen-binding molecule according to the methods of the present disclosure, the subject shows observable and/or measurable reduction in or absence of one or more of the following: reduction in the number of infected cells or absence of the infected cells; reduction in the percent of total cells that are infected; relief to some extent of one or more of the symptoms associated with the specific infection (e.g., symptoms associated with hMPV infection); reduced morbidity and mortality, and/or improvement in quality of life issues. The above parameters for assessing successful treatment and improvement in the disease are readily measurable by routine procedures familiar to a physician.

In some embodiments, the subject has or is at risk of developing an hMPV infection.

In some embodiments, the subject is a mammal. In some embodiments, the subject is a human.

In some embodiments, the subject is susceptible to viral infection (e.g., hMPV).

In some embodiments, the subject is an elderly subject.

In some embodiments, the antigen-binding molecule of the present disclosure is administered by intramuscular injection, intravenous injection, or subcutaneous injection.

### Methods for Detection

Antigen-binding molecules of the present disclosure may be used in various methods of detecting, including those described herein and others known in the art, for example, methods of detecting an antibody in contact with an hMPV F protein or a cell that expresses an hMPV F protein on its surface. Immunoassays useful in the practice of the methods disclosed herein include fluorescence activated cell sorting (FACS) analysis, enzyme-linked immunosorbent assay (ELISA), immunoprecipitation or radioimmunoprecipitation (RIA).

These methods may be carried out in vivo, ex vivo, or in vitro. In particular, the step of contacting an antibody with an hMPV F protein or with a cell that expresses an hMPV F protein on its surface may be carried out in vivo, ex vivo, or in vitro. The methods may be carried out in a cell-based or a cell-free system.

Potential hMPV F protein antigen-binding molecules may be evaluated in vivo, such as, in an animal model. In such an in vivo model, the effects of the antigen-binding molecules may be evaluated in the circulation (e.g., blood) or heart, or in other organs such as lung, liver, kidney, or the brain.

Provided is a method for detecting antibodies specific to an hMPV F protein, the method comprising:
a.) contacting a biological sample with an hMPV F protein; and
b.) contacting an epitope-specific antigen-binding molecule according to the present disclosure with the hMPV F protein.

In some embodiments, the hMPV F protein is coated on a microwell plate prior to contact with the biological sample or antigen-binding molecule.

In some embodiments, a half maximal effective concentration (EC₅₀) of anti-hMPV F protein antibodies in the biological sample is determined by calculating the reciprocal dilution of biological sample at which 50% of the binding of the antigen-binding molecule is inhibited.

In some embodiments, the biological sample is serum.

For example, provided is a method for detecting a titer of serum anti-hMPV F protein antibodies competing with a biotinylated anti-hMPV F protein antibody for binding to an hMPV F antigen. The method comprises:
a. coating an ELISA plate with hMPV F antigen to prepare an hMPV F antigen-coated ELISA plate;
b. adding serum to the hMPV F antigen-coated ELISA plate;
c. adding the biotinylated anti-hMPV F protein antibody to the hMPV F antigen-coated ELISA plate under conditions effective to allow binding of the biotinylated anti-hMPV F protein antibody to the hMPV F antigen-coated ELISA plate in the presence of the serum;
d. detecting the binding of the biotinylated anti-hMPV F protein antibody to the hMPV F antigen-coated ELISA plate in the presence of the serum using a streptavidin polypeptide that binds to the biotinylated anti-hMPV F protein antibody and triggers color development that is proportional to the amount of biotinylated anti-hMPV F protein antibody bound to the hMPV F antigen; and
e. identifying the titer of serum anti-hMPV F protein antibodies that effectively compete with the biotinylated anti-hMPV F protein antibody by identifying the reciprocal dilution of serum at which 50% of the binding of the biotinylated anti-hMPV F protein antibody is inhibited.

The present method contemplates any anti-hMPV F protein antibody that binds to hMPV F protein. In some embodiments, the method detects the titer of serum anti-hMPV F protein antibodies competing with a biotinylated 13E10 hMPV F protein epitope antibody. In some embodiments, the method detects the titer of serum anti-hMPV F protein antibodies competing with a biotinylated 42C2 hMPV F protein epitope antibody. In some embodiments, the method detects the titer of serum anti-hMPV F protein antibodies competing with a biotinylated 17D10 hMPV F protein epitope antibody.

Antigen-binding molecules of the present disclosure are contemplated for use in the determination of the identity of a recombinantly produced hMPV F protein.

The anti-hMPV F protein antibody of the present disclosure that binds the 13E10 epitope anti-hMPV F protein antibody only binds hMPV F protein in the pre-fusion confirmation. As such, the 13E10 epitope anti-hMPV F protein antibody is contemplated in the present methods to be used for evaluating the stability of a pre-fusion stabilized hMPV F protein. In some embodiments, the anti-hMPV F protein antibody that binds the 13E10 epitope may be used to determine the hMPV F protein conformation because it only binds hMPV F protein in the pre-fusion confirmation.

The anti-hMPV F protein antibody of the present disclosure that binds the 17D10 epitope anti-hMPV F protein antibody binds hMPV F protein in the pre-fusion and post-fusion confirmations.

The anti-hMPV F protein antibody of the present disclosure that binds the 42C2 epitope anti-hMPV F protein antibody only binds hMPV F protein in the pre-fusion and post-fusion confirmations.

The present method contemplates a binding assay with a 13E10 epitope anti-hMPV F protein antibody and either 42C2 epitope or 17D10 epitope anti-hMPV F protein antibody to evaluate a post-fusion conformation of an hMPV F protein because 42C2 epitope and 17D10 epitope anti-hMPV F protein antibodies bind a post-fusion conformation of hMPV F protein but 13E10 epitope anti-hMPV F protein antibody does not bind a post-fusion conformation of hMPV F protein.

For example, in a binding assay with a 13E10 epitope anti-hMPV F protein antibody and either 42C2 epitope or 17D10 epitope anti-hMPV F protein antibody, ratios of the binding to the anti-hMPV F protein antibodies may be used to evaluate conformation of the hMPV F protein. Without wishing to be bound by theory, a decrease in 13E10 epitope anti-hMPV F protein antibody binding but no change in binding of 42C2 epitope or 17D10 epitope anti-hMPV F protein antibodies may indicate a transition from pre-fusion to post-fusion conformation of the hMPV F protein.

The present method contemplates an assay to evaluate the conformation of an hMPV F protein vaccine antigen, the method comprising contacting the epitope-specific antigen-binding molecules of the present disclosure with the vaccine antigen. For example, a 13E10 epitope anti-hMPV F protein antibody and either 42C2 epitope or 17D10 epitope anti-hMPV F protein antibody may be used in a binding assay of the present disclosure to evaluate conformation of hMPV F protein antigen. In a further example, in the production of a pre-fusion conformation hMPV F protein antigen, the 13E10 epitope anti-hMPV F protein antibody only binds hMPV F protein in the pre-fusion confirmation, while the 17D10 epitope and 42C2 epitope anti-hMPV F protein antibodies bind hMPV F protein in the pre-fusion and post-fusion confirmations. Without wishing to be bound by theory, conformational selectivity of the epitope-specific antibodies of the present disclosure allows evaluation of the conformation of an hMPV vaccine antigen.

In some embodiments, the pre-fusion conformation confers stability to an hMPV protein relative to a post-fusion conformation. Illustrative antibody sequences of the present disclosure are shown in Table 5.

TABLE 5
Illustrative antibody sequences of the present disclosure

| SEQ ID | | |
|---|---|---|
| NO: | Description | Sequence |
| 26 | 13E10 | ATGGAAAACAAAATGGATTTTCAGATGCAGATTATCAGC |
| | mlgK | TTGCTGCTAATCAGTGTCACAGTCATAGTGTCTACTGGA |
| | | GAAATTGTGCTCACCCAGTCTCCGACCACCATGGCTGCA |
| | | TCTCCCGGGGAGAAGATCACTATCACCTGCAGTGCCAGC |
| | | TCAAGTATAAGTTCCAATTACTTACATTGGTATCAGCAG |
| | | AAGGCAGGATTCTCCCCTAAACTCTTGATTTATAGGACA |
| | | TCCAATCTGGCTTCTGGAGTCCCAGCTCGCTTCAGTGGC |
| | | AGTGGGTCTGGGACCTCTTACTCTCTCACAATTGGCACC |
| | | ATGGAGGCTGAAGATGTTGCCACTTACTACTGCCAGCAG |
| | | GGTAGTAGTCTACCACGCACGTTCGGAGGGGGGACCAA |
| | | GCTGGAAATAAAACGGGCTGATGCTGCACCAACTGTATC |
| | | CATCTTCCCACCATCCAGTGAGCAGTTAACATCTGGAGG |
| | | TGCCTCAGTCGTGTGCTTCTTGAACAACTTCTACCCCAGA |
| | | GACATCAATGTCAAGTGGAAGATTGATGGCAGTGAACG |
| | | ACAA |
| 27 | 13E10 | MENKMDFQMQIISLLLISVTVIVSTG |
| | mIgK ss | |
| 28 | 13E10 | EIVLTQSPTTMAASPGEKITITCSASSSISSNYLHWYQQKAG |
| | IGKV | FSPKLLIYRTSNLASGVPARFSGSGSGTSYSLTIGTMEAEDV |
| | | ATYYCQQGSSLPRTFGGGTKLEIK |
| 29 | 13E10 | RADAAPTVSIFPPSSEQLTSGGASVVCFLNNFYPRDINVKW |
| | IGKC | KIDGSERQ |
| 30 | 13E10 | ATGAAGTTGTGGCTGAACTGGATTTTCCTTGTAACACTTT |
| | mlgG1 | TAAATGGTATCCAGTGTGAGGTGAAGCTGGTGGAGTCTG |
| | | GAGGAGGCTTGGTACAGCCTGGGGGTTCTCTGAGTCTCT |
| | | CCTGTGCAGCTTCTGGATTCACCTTCACTGATTACTACAT |
| | | GAACTGGGTCCGCCAGCCTCCAGGGAAGGCACTTGAGTG |
| | | GTTGGGCTTTATTAGAAACAAAGATAATGGTTACACAAC |
| | | AGAGTATAGTGCATCTGTGAAGGGTCGGTTCACCATCTC |
| | | CAGAGATAATTCCCAAAGCATCCTCTATCTTCAAATGAA |
| | | TGCCCTGAGAGCTGAGGACAGTGCCACTTATTACTGTAC |
| | | AAGATATTACTTTGGTTACGACGGGGACTACTTTGACTA |
| | | CTGGGGCCAAGGCACCACTCTCACAGTCTCCTCAGCCAA |
| | | AACGACACCCCCATCTGTCTATCCACTGGCCCCTGGATC |
| | | TGCTGCCCAAACTAACTCCATGGTGACCCTGGGATGCCT |
| | | GGTCAAGGGCTATTTCCCTGAGCCAGTGACAGTGACCTG |
| | | GAACTCTGGATCCCTGTCCAGCGGTGTGCACACCTTCCC |
| | | AGCT |
| 31 | 13E10 | MKLWLNWIFLVTLLNGIQC |
| | mIgG1 ss | |
| 32 | 13E10 | EVKLVESGGGLVQPGGSLSLSCAASGFTFTDYYMNWVRQP |
| | IGHV | PGKALEWLGFIRNKDNGYTTEYSASVKGRFTISRDNSQSIL |
| | | YLQMNALRAEDSATYYCTRYYFGYDGDYFDYWGQGTTLT |
| | | VSS |
| 33 | 13E10 | AKTTPPSVYPLAPGSAAQTNSMVTLGCLVKGYFPEPVTVT |
| | IGHG1 | WNSGSLSSGVHTFPA |
| 34 | 17D10 | ATGTCCTCTGCTCAGTTCCTTGGTCTCCTGTTGCTCTGTTT |
| | mIGK = | TCAAGGTGCCAGATGTGATATCCAGCTGACACAGACTAC |
| | 17E10 | ATCCTCCCTGTCTGCCTCTCTGGGAGACAGAGTCACCAT |
| | | CAGTTGCAGGGCAAGTCAGGACATTAGCAATTATTTAAA |
| | | CTGGTATCAGCAGAAACCAGATGGAACTGTTAAACTCCT |
| | | GATCTCCTACACATCAGGATTACACTCAGGAGTCCCATC |
| | | AAGGTTCAGTGGCAGTGGGTCTGGAACAGATTATTCTCT |
| | | CACCATTAGCAACCTGGAGCAAGAAGATATTGCCACTTA |
| | | CTTTTGCCAACAGGGTAATACGCTTCCGTGGACGTTCGG |
| | | TGGAGGCACCAAGCTGGAAATCAAACGGGCTGATGCTG |
| | | CACCAACTGTATCCATCTTCCCACCATCCAGTGAGCAGT |
| | | TAACATCTGGAGGTGCCTCAGTCGTGTGCTTCTTGAACA |
| | | ACTTCTACCCCAGAGACATCAATGTCAAGTGGAAGATTG |
| | | ATGGCAGTGAACGACAA |
| 35 | 17D10 | MSSAQFLGLLLLCFQGARC |
| | mIGK = | |
| | 17E10 | |
| | signal | |
| | sequence | |
| 36 | 17D10 | DIQLTQTTSSLSASLGDRVTISCRASQDISNYLNWYQQKPD |
| | mIGK = | GTVKLLISYTSGLHSGVPSRFSGSGSGTDYSLTISNLEQEDIA |
| | 17E10 | TYFCQQGNTLPWTFGGGTKLEIK |
| | IGKV | |
| 37 | 17D10 | RADAAPTVSIFPPSSEQLTSGGASVVCFLNNFYPRDINVKW |
| | mIGK = | KIDGSERQ |
| | 17E10 | |
| | IGKC | |
| 38 | 17D10 | ATGGGATGGAGCTGTATCATCCTCATTTTGGTAGCAGCA |
| | mIGHG1 = | GCTACAGGTGTCCACTCCCAGGTCCAACTGCAGCAGCCT |
| | 17E10 | GGGGCTGAGCTTGTGAAGCCTGGGGCTTCAGTGAAGATG |
| | | TCCTGCAAGGCTTCTGGCTACACCTTCACCAGCTACTGG |
| | | ATAACCTGGGTGAAGCAGAGGCCTGGACAGGGCCTTGA |
| | | GTGGATTGGAGATATTTATCCTGGTAGTGGTAGTATTAC |
| | | CTACAATGAGAAGTTCAAGACCAAGGCCACACTGACTGT |
| | | AGACACATCCTCCAGCACAGCCTACATGCAGCTCAACAG |
| | | CCTGACATCTGAGGACTCTGCGGTCTATTACTGTGCAGG |
| | | ATTACTACGGTTAACCTTCGATGTCTGGGGCACAGGGAC |
| | | CACGGTCACCGTCTCCTCAGCCAAAACGACACCCCCATC |
| | | TGTCTATCCACTGGCCCCTGGATCTGCTGCCCAAACTAA |
| | | CTCCATGGTGACCCTGGGATGCCTGGTCAAGGGCTATTT |
| | | CCCTGAGCCAGTGACAGTGACCTGGAACTCTGGATCCCT |
| | | GTCCAGCGGTGTGCACACCTTCCCAGCT |
| 39 | 17D10 | MGWSCIILILVAAATGVHS |
| | mIGHG1 = | |
| | 17E10 | |
| | signal | |
| | sequence | |
| 40 | 17D10 | QVQLQQPGAELVKPGASVKMSCKASGYTFTSYWITWVKQ |
| | mIGHGI = | RPGQGLEWIGDIYPGSGSITYNEKFKTKATLTVDTSSSTAY |
| | 17E10 | MQLNSLTSEDSAVYYCAGLLRLTFDVWGTGTTVTVSS |
| | IGHV | |
| 41 | 17D10 | AKTTPPSVYPLAPGSAAQTNSMVTLGCLVKGYFPEPVTVT |
| | mIGHG1 = | WNSGSLSSGVHTFPA |
| | 17E10 | |
| | mIGHG1 | |
| 42 | 42C2_mIGK | ATGGAGACACATTCTCAGGTCTTTGTATACATGTTGCTGT |
| | | GGTTGTCTGGTGTTGAAGGAGACATTATGATGACCCAGT |
| | | CTCACAAATTCATGTCCACATCAGTAGGAGACAGGGTCA |
| | | TTTTCACCTGCAAGGCCAGTCAGGATGTGGGTACTGCTG |
| | | TAGCCTGGTATCAACAGAAATCAGGGCAATCTCCTAAAC |
| | | TACTGATTTACTGGGCATCCACCCGGCACACTGGAGTCC |
| | | CTGATCGCTTCACAGGCAGTGGATCTGGGACAGATTTCA |
| | | CTCTCACCATTAGCAATGTGCAGTCTGAAGACTTGGCAG |
| | | ATTATTTCTGTCAACAATATACCAGCTATCCTCTCACGTT |
| | | CGGTGCTGGGACCAAGCTGGAGCTGAAACGGGCTGATG |
| | | CTGCACCAACTGTATCCATCTTCCCACCATCCAGTGAGC |
| | | AGTTAACATCTGGAGGTGCCTCAGTCGTGTGCTTCTTGA |
| | | ACAACTTCTACCCCAGAGACATCAATGTCAAGTGGAAGA |
| | | TTGATGGCAGTGAACGA |
| 43 | 42C2 mIGK | METHSQVFVYMLLWLSGVEG |
| | signal | |
| | sequence | |
| 44 | 42C2 mIGK | DIMMTQSHKFMSTSVGDRVIFTCKASQDVGTAVAWYQQK |
| | variable | SGQSPKLLIYWASTRHTGVPDRFTGSGSGTDFTLTISNVQSE |
| | sequence | DLADYFCQQYTSYPLTFGAGTKLELK |
| 45 | 42C2 | RADAAPTVSIFPPSSEQLTSGGASVVCFLNNFYPRDINVKW |
| | mIGK | KIDGSER |
| | mIGKC | |
| 46 | 42C2 | ATGGGCAGGCTTACTTCTTCATTCCTGTTACTGATTGTCC |
| | mIGHG | CTGCATATGTCCTGTCCCAGGTTACTCTGAAAGAGTCTG |
| | | GCCCTGGGATATTGCAGCCCTCCCAGACCCTCAGTCTGA |
| | | CTTGTTCTTTCTCTGGGTTTTCACTGAGCACTTTTGGTAT |
| | | GGGTGTAGTCTGGACTCGTCAGCCTTCAGGGAAGGGTCT |
| | | GGAGTGGCTGGCACACATTTGGTGGGATGATGATAATTA |
| | | CTATAACCCAGCCCTGAAGAGTCGGCTCACAATCTCCAA |
| | | GGATACCTCCAAAAACCAGGTATTCCTCAAAATCGCCAA |
| | | TGCGGACACTGCAGATACTGCCACATACTACTGTGCTCG |
| | | AATAGTTAAGGTCCTGGAACAGTACTTCGATGTCTGGGG |
| | | CACAGGGACCACGGTCACCGTCTCCTCAGCCAAAACGAC |
| | | ACCCCCATCTGTCTATCCACTGGCCCCTGGATCTGCTGCC |
| | | CAAACTAACTCCATGGTGACCCTGGGATGCCTGGTCAAG |
| | | GGCTATTTCCCTGAGCCAGTGACAGTGACCTGGAACTCT |
| | | GGATCCCTGTCCAGTGGTGTG |
| 47 | 42C2 | MGRLTSSFLLLIVPAYVLS |
| | mIGHG | |
| | signal | |
| | sequence | |
| 48 | 42C2 | QVTLKESGPGILQPSQTLSLTCSFSGFSLSTFGMGVVWTRQP |
| | mIGHG | SGKGLEWLAHIWWDDDNYYNPALKSRLTISKDTSKNQVFL |
| | mIGHV | KIANADTADTATYYCARIVKVLEQYFDVWGTGTTVTVSS |
| 49 | 42C2 | AKTTPPSVYPLAPGSAAQTNSMVTLGCLVKGYFPEPVTVT |
| | mIGHG | WNSGSLSSGV |
| | mIGHG1 | |

### EXAMPLES

### Example 1: hMPV F Protein Antibody Generation

Neutralizing monoclonal antibodies to the hMPV F protein, specifically hMPV033, were generated and characterized from mice immunized with an hMPV F protein fragment (hMPV008) fused to I53-50A (as described in WO 2019/169120 A1) and screened for antibodies binding to hMPV033 but not binding to 153-50A, RSV F protein, or hexahistine (his6) tag.

Antibodies that bound specifically to hMPV F protein were tested in a virus neutralization assay and a total of 24 neutralizing antibodies were identified. Three neutralizing antibodies, 17D10 hIgG1, 13E10 hIgG1, and 42C2 hIgG1, were humanized, expressed, and purified and the epitopes of each antibody mapped by CovalX™. The three illustrative antibodies have the following characteristics:
1. Bind to hMPV F protein but not RSV F protein
2. Neutralizing antibodies
3. Bind non-linear, conformation dependent epitopes
4. Antibodies 17D10 and 42C2 bind both pre- and post-fusion conformations of hMPV F protein and 13E10 antibody binds only the pre-fusion conformation of hMPV F protein.

The hMPV008 antigen contains the ectodomain (1-472 AA) of the NIH V4-B mutant fused to virus-like particle 153-50A with a 16 residue GS linker. V4-B was constructed with a B strain sequence and is a single chain construct with a 6 amino acid Gly linker replacing the F1/F2 cleavage site. The mutant also contains 6 Cys mutations (A63C, A140C, A147C, K188C, K450C and S470C). Amino acid 63C forms a disulfide bond with the natural occurring Cys in position 60C and amino acid 188C forms a disulfide bond with the natural occurring Cys in position 182C. The 60C-63C bond is an intraprotomer bond, while the 182-188 bond is an interprotomer bond.

The hMPV033 antigen contains the ectodomain (1-472 AA) of the UT-A CL-28 mutant fused to 153-50A with a 16 residue GS linker. CL-28 was constructed in the A strain sequence with a 6 amino acid Gly linker replacing the F1/F2 cleavage site. The mutant also contains the following mutations: T127C, N153C, A185P, V231I, L219K, G294E, T365C, and V463C. In some embodiments, the mutant contains 368N. In some embodiments, the mutant contains T127C, N153C, A185P, V231I, L219K, G294E, T365C, V463C, and H368N. In some embodiments, the mutant contains 368H. 127C-153C and 365C-463C form intraprotomer disulfide bonds.

hMPV008 and hMPV033 Antigen Generation

Expi293 cells in log phase growth were counted and seeded in 220 ml at 2.5×10⁶ cells/ml in each of four 1 L flasks (total volume 880 ml). Cells were incubated overnight at 36° C. with shaking (120 rpm). The next day the cells were counted and diluted to 3×10⁶ cells/ml in 235 ml per 1 L flask. Cells were transiently transfected as follows. 1000 μg plasmid DNA was diluted to 35 ml final volume with OptiMEM and gently mixed. 2.5 ml of Transporter 5 transfection reagent was diluted to a final volume of 35 ml with OptiMEM and gently mixed. The Diluted Transporter 5 was added to the diluted DNA, mixed, incubated at room temperature for 10 minutes and then 17.5 ml added dropwise to each 1 L flask while gently swirling the flask. Cells were placed back in the incubator, shaking for 4 days.

### Purification

Constructs were purified directly from conditioned Expi293F expression media by Immobilized Metal Affinity Chromatography (IMAC). INDIGO Ni-Agarose was washed with 5 column volumes (CV) of water followed by 5 CV of equilibration buffer, then resuspended in 1-2 CV of equilibration buffer. Supernatants were clarified by centrifugation at 4,000×g followed by filtration using 0.2 or 0.45 μm vacuum filtration units. Resin suspension was added to the supernatant such that 2-3 mL of resin was used per liter of supernatant. The supernatant-resin slurry was stirred gently at 4° C. for 2-3 hours, then the resin was collected by filtration using a 0.45 μm vacuum filter and transferred to a gravity column using equilibration buffer. The resin was washed with 20 CV wash buffer, and proteins were eluted using 5-10 CV elution buffer.

### ELISA Assay and Octet Assay—2^{nd} Screen

For screening the second round of 117 clones against hMPV F protein, a capture ELISA was used. High binding plates were coated overnight at 4° C. with 100 ng/well of hMPV033 in 100 mM sodium carbonate-bicarbonate buffer, pH 9.6. The following day, plates were washed 3 times with Wash Buffer (1×PBS, 0.05% Tween-20, pH 7.4) and blocked with 150 μL/well of Block Buffer (Wash Buffer with 1% BSA) for 1 hour at room temperature. Supernatant samples were thawed at room temperature, then inverted several times to mix. Each sample was diluted 10-fold using Block Buffer as diluent, followed by additional dilutions for 50-fold and 500-fold final. Samples were loaded at 100 μL/well and the plates incubated covered at room temperature without agitation for 1 hour. Samples were aspirated and the plates were washed 5 times with Wash Buffer, then goat anti-mouse HRP-conjugated antibody diluted 1:5,000 in Block Buffer was added at 100 μL/well, and the plates incubated covered at room temperature without agitation for 1 hour. Conjugate was aspirated and the plates were washed 5 times with wash buffer, then 100 μL/well TMB was applied and incubated for approximately 5 minutes in the dark at room temperature. Reaction progress was stopped with 100 μL/well of 0.6 N sulfuric acid and the absorbance at 450 nm was measured within ten minutes. The endpoint absorbance values of the 50 and 500-fold dilutions were used to rank mAb binding. The top 92 highest binding samples were then screened for binding by Biolayer Interferometry (BLI). BLI on an Octet Red96 was carried out in the following manner: anti-pentaHis biosensors were dipped in assay buffer for 30 sec to achieve a baseline. hMPV033 in assay buffer at 10 μg/mL was loaded onto the sensors for 30 sec, followed by an additional baseline step. Next, the sensors were dipped into each supernatant for 60 sec to observe binding of the antibodies to hMPV033. The shift at 120 sec was used to rank binding (data not shown).

### Immunization

Five SJL female mice (4-6 weeks old) were immunized with hMPV008-I53-50A immunogen in “pre-fusion” conformation. The immunogen contained a C-terminal His6 tag and was produced from a stable miCHO K1 cell line. Serum samples from immunized mice collected and tested for titers against hMPV033 to avoid detection of antibodies to I53-50A (as described in WO 2019/169120 A1). A total of 42 supernatants evaluated by ELISA resulted in a positive signal with hMPV033 and a negative signal with I53-50A (data not shown).

### Neutralizing Assay of 1st Screen Materials

The 42 supernatants were evaluated in an hMPV/A neutralization assay.

All supernatants were diluted 1:8 initially, followed by two-fold serial dilutions. Neutralizing antibody titers were defined as the final dilution at which there was a 50% reduction in viral cytopathic effect (CPE). The final serum dilution is the final dilution of the serum without taking into account the additional four-fold dilution that occurs with mixing of the diluted serum (50 μl) to the virus (50 μl) and then the addition of cells (100 μl).

All the monoclonal antibodies were provided at a concentration of 200 μg/ml and were subsequently diluted 1:8 initially, followed by two-fold serial dilutions. To calculate the inhibitor concentration 50% (IC50), the end titer is converted into a dilution factor. The dilution factor is used to divide the original monoclonal antibody concentration to determine the inhibitor IC50. A further four-fold dilution is used to obtain the final serum dilution for calculating the IC50. Table 6 shows the results of neutralizing antibody titer (without 4-fold dilution adjustment), the original IC50 (without 4-fold dilution adjustment) and the Final IC50 (with 4-fold dilution adjustment).

For example, monoclonal antibody 37H4 mAB 0.2 mg/mL gave a neutralizing antibody titer of 9 log₂ which is a dilution factor of 512. 200 micrograms÷512=390.6 ng/ml. A further 4-fold reduction to account for the dilution of the virus and cell volume results in a final concentration of 97.7 ng/ml.

The background signal in the assay is 2.0 log₂ and any result of 5.0 log₂ or higher was considered positive. Seven antibodies (3G3, 19E9, 8H11, 17D10, 17E10, 13E10 and 18D2) were demonstrated to have neutralizing activity above 5.0 log₂ (Table 6). One of the 7 clones, 18D2, had cross-reactivity and was not pursued. The remaining 6 antibodies were scaled up for antibody production and purification. An hMPV/A neutralization assay with 6 purified antibodies (3G3, 19E9, 8H11, 17D10, 17E10, and 13E10) was performed. The data for all 6 antibodies demonstrated neutralizing activity (background 2.0 log₂) with titers of 8.0 to 12.5 log 2 (data not shown).

73 additional supernatants were identified by BLI as binding hMPV033 but not I53-50A as described above. The 73 supernatants were evaluated in an hMPV/A neutralization assay and eighteen Abs (30G3, 42C2, 30G4, 51D9, 40A9, 37H4, 35F12, 37E1, 27F1, 50B10, 32H7, 39A4, 30D7, 41E5, 56B4, 22B2, 39D8, and 32G3) were demonstrated to have neutralizing activity above 5.0 log₂ (background 2.0 log₂) (data not shown). Four antibodies (37H4, 42C2, 30G3, and 56B4) were scaled up for antibody production and purification. An hMPV/A neutralization assay with the 4 purified antibodies was performed. The data for all 4 antibodies demonstrated neutralizing activity (background 2.0 log₂) with titers of 6.5 to 10 log 2 (Table 6). Following these results, recombinant versions of antibody candidates 19E9, 17D10, 13E10, and 42C2 were produced with human IgG1 Fc.

TABLE 6

| hMPV/A RL Bx | | | Inhibitory concentration | |
|---|---|---|---|---|
| Microneutralization Assay | | | 50% (IC50) | |
| | | hMPV/A | Original | Final |
| | | RL | serum | serum |
| | Experiment | Bx Titer | dilution | dilution |
| Aliquot Name | # | (Log 2) | (ng/mL) | (ng/mL) |
| 37H4 mAB 0.2 | 3 | 9.0 | 390.6 | 97.7 |
| mg/mL | | | | |
| 56B4 mAB 0.2 | 3 | 10.0 | 195.3 | 48.8 |
| mg/mL | | | | |
| 42C2 mAB 0.2 | 3 | 9.0 | 390.6 | 97.7 |
| mg/mL | | | | |
| 30G3 mAB 0.2 | 3 | 6.5 | 2209.7 | 552.4 |
| mg/mL | | | | |
| Human 17D10 mAB | 3 | 10.5 | 138.1 | 34.5 |
| 0.2 mg/mL | | | | |
| Mouse 17D10 mAB | 3 | 10.0 | 195.3 | 48.8 |
| 0.2 mg/mL | | | | |
| 13E10 (mouse) | 4 | 8.0 | 781.3 | 195.3 |
| (200 μg/mL) | | | | |
| 13E10 hIgG1 | 4 | 9.0 | 390.6 | 97.7 |
| (200 μg/mL) | | | | |
| 42C2 (mouse) | 4 | 9.0 | 390.6 | 97.7 |
| (200 μg/mL) | | | | |
| 42C2 hIgG1 | 4 | 9.5 | 276.2 | 69.1 |
| (200 μg/mL) | | | | |

Confirmation of Neutralizing Activity, Specificity, Bin, and K_{D} of humanized Abs

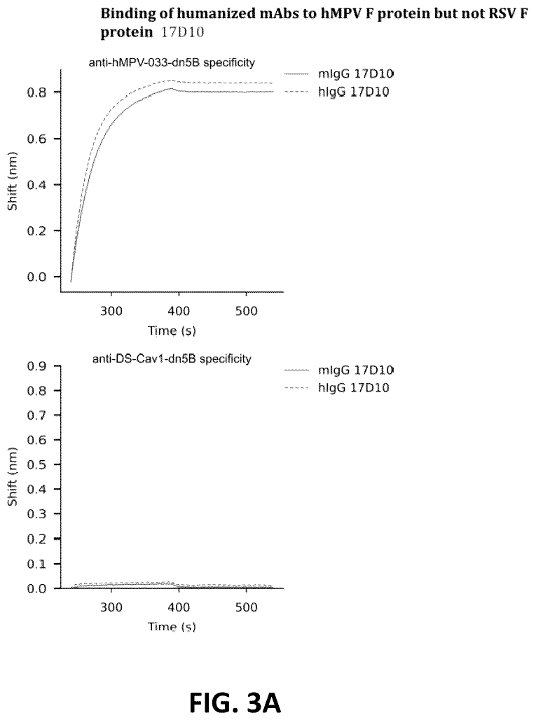

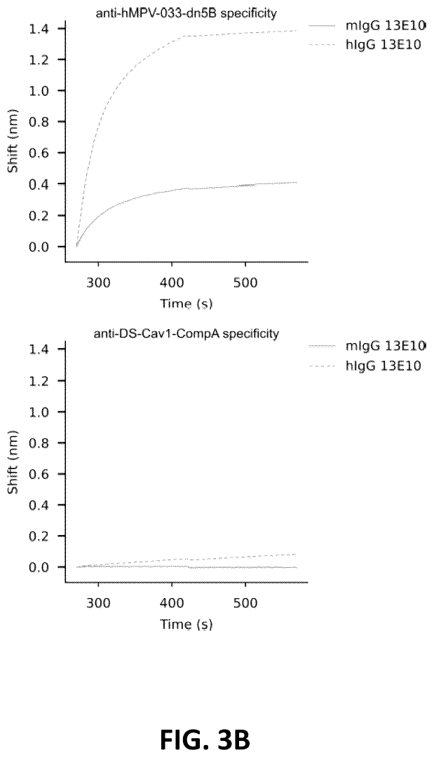

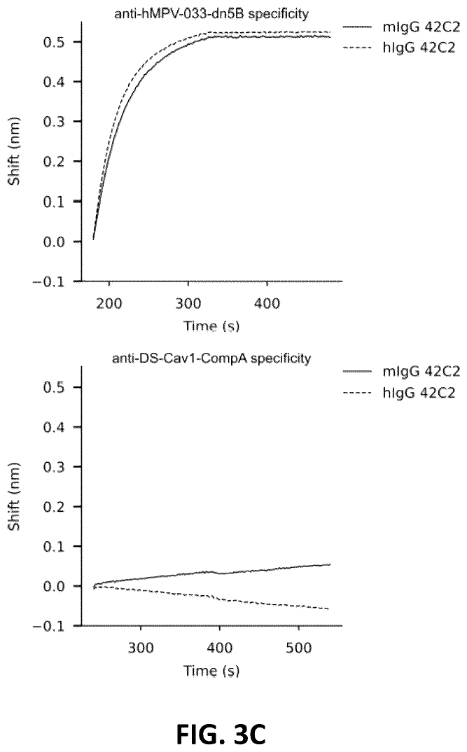

The recombinant humanized antibodies (171D10 hIgG1, 13E10 hIgG1, and 42C2 hIgG1) were tested to confirm hMPV F protein specificity, neutralization, binning, and K_{D} estimation. All 3 humanized antibodies were confirmed to be potent neutralizing antibodies (Table 6). Binding to hMPV F protein but not RSV F protein was confirmed by Octet with all three humanized antibodies (FIGS. 3A-3C). To estimate the affinity of each humanized antibody for hMPV033, I53-50A-hMPV033 was immobilized on anti-pentaHis biosensors and then dipped into a dilution series of each humanized neutralizing antibody. The estimated K_{D} values ranged from 4.7 to 30 nM (data not shown).

### Epitope Mapping

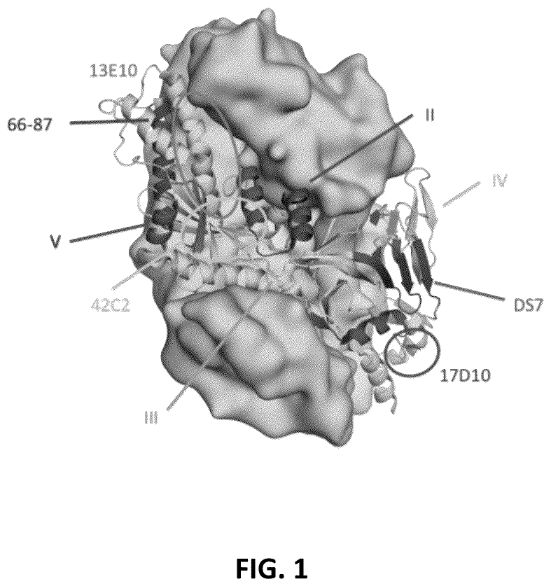

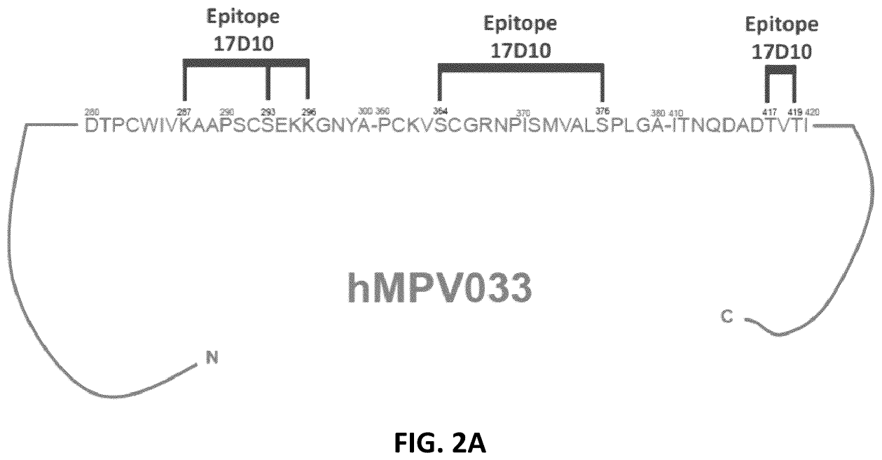

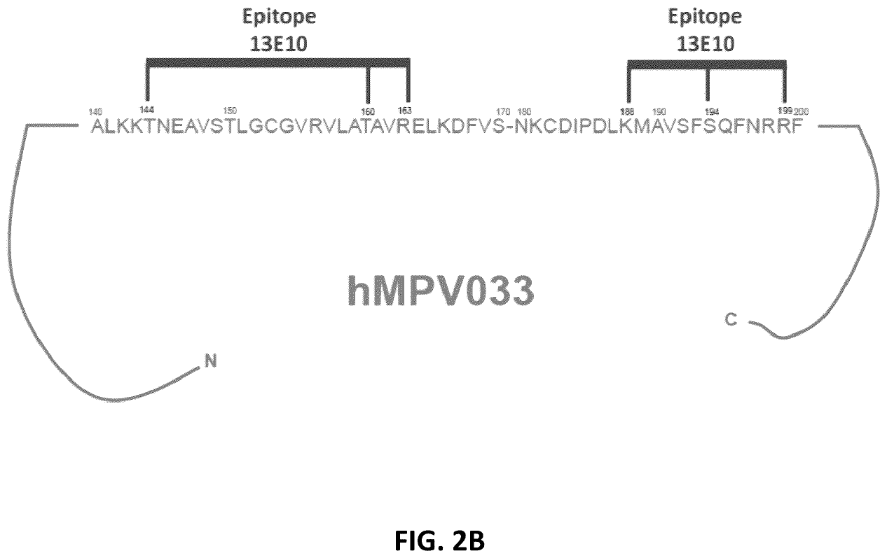

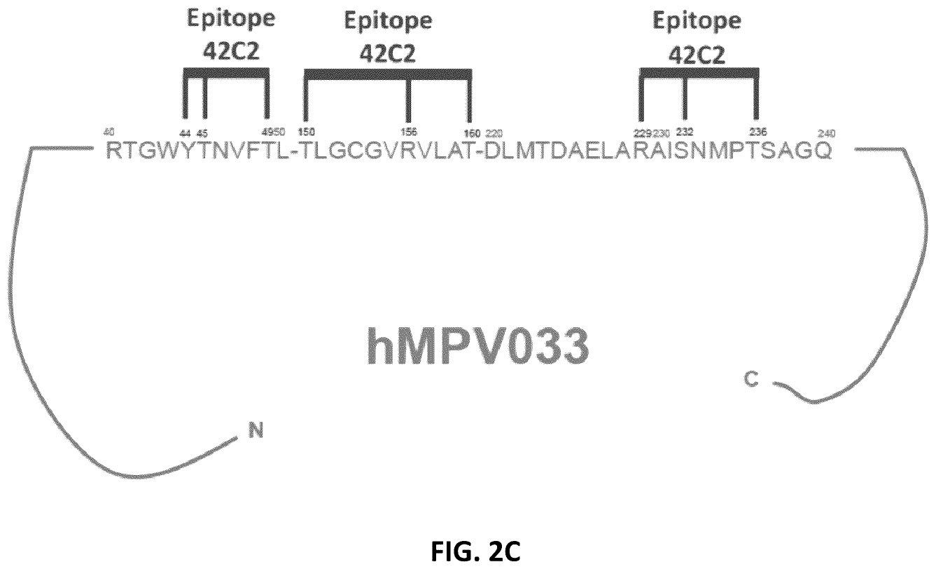

Three monoclonal antibodies (171D10 hIgG1, 13E10 hIgG1, and 42C2 hIgG1) for epitope mapping. CovalX™ identified the epitopes recognized by each antibody by crosslinking the antigen-antibody complex, multi-enzymatic proteolysis followed by nLC-Orbitrap MS-MS analysis and demonstrated that each antibody recognizes non-linear epitopes (FIGS. 2A-2C). A graphic representation of the epitopes as well as other antigenic sites for neutralizing antibodies to hMPV F protein are shown in FIG. 1.

2D class averaging of nsEM images of 153-50A-hMPV033 indicated the F proteins are in the pre-fusion conformation and a mixture of compact trimers and open trimers.

### Characterization of the Molecular Interfaces

To determine the epitopes of the hMPV033/17D10 hlgG1, hMPV033/13E10 hlgG1 and hMPV033/42C2 hlgG1 complexes with high resolution, each protein complex was incubated with deuterated cross-linkers and subjected to multi-enzymatic cleavage. After enrichment of the crosslinked peptides, the samples were analyzed by high resolution mass spectrometry (nLC-Q-Exactive MS) and the data generated was analyzed using XQuest™ and Stavrox™ software.

For this analysis, nano-liquid chromatography (nLC) in combination with Q-Exactive MS Analysis were used with the following parameters:

### Ultimate 3000-RSLC

A 98/02/0.1 H2O/ACN/HCOOH v/v/v
B 20/80/0.1 H2O/ACN/HCOOH v/v/v
gradient 4-55% B in 33 minutes
injected volume 1 μl
precolumn 300-μm ID×5-mm C18 PepMap™
precolumn flow rate 50 μl/min
column 75-μm ID×15-cm C18 PepMapRSLC
column flow rate 300 nl/min

### Mass Spectrometry: Q-Exactive MS Analysis

The Q-Exactive MS analysis was performed with the following parameters:
Scan type: full MS
Scan range: 350-1600 m/z
Resolution: 70,000
μscan: 1
Max injection time: 100 ms
Spray voltage: 1.7 kV
Capillary voltage: 275° C.
S-lens RF level: 55.0
AGC target: 3e6
Default charge state: 2
   dd-MS²
Resolution: 17,500
AGC target: 1e5
Ion isolation window: 4 m/z units
Max injection time: 50 ms
Normalized collision energy: 30%
Loop count: 5
Dynamic exclusion: ON
Dynamic exclusion params: 30.0 s
Minimum AGC target: 8e3
Intensity threshold: 1.6e5

### Reduction Alkylation

20 μL of the hMPV033/Antibodies mixtures were mixed with 2 μL of DSS d0/d12 (2 mg/mL; DMF) for 180 minute incubation at room temperature. After incubation, reaction was stopped by adding 1 μL of ammonium bicarbonate (20 mM final concentration) for 1 hour incubation time at room temperature. Then, the solution was dried using a SpeedVac followed by H₂O 8M urea suspension (20 μL). After mixing, 2 μl of DTT (500 mM) were added to the solution. The mixture was then incubated for 1 hour at 37° C. After incubation, 2 μl of iodoacetamide (1M) were added and incubated for 1 hour at room temperature, in a dark room. After incubation, 80 μl of the proteolytic buffer were added. The trypsin buffer contains 50 mM Ambic pH 8.5, 5% acetonitrile; the Chymotrypsin buffer contains Tris HCl 100 mM, CaCl₂) 10 mM pH 7.8; the ASP-N buffer contains Phosphate buffer 50 MM pH 7.8; the elastase buffer contains Tris HCl 50 mM pH 8.0; and the thermolysin buffer contains Tris HCl 50 mM, CaCl₂) 0.5 mM pH 9.0.

### Trypsin Proteolysis

100 μl of the reduced/alkyled hMPV033/17D10_hlgG1, hMPV033/13E10_hlgG1 or hMPV033/42C2_hlgG1 mixtures were mixed with 1.24 μl of trypsin (Promega) with the ratio 1/100. The proteolytic mixtures were incubated overnight at 37° C.

### Chymotrypsin Proteolysis

100 μl of the reduced/alkyled hMPV033/17D10 hlgG1, hMPV033/13E10_hlgG1 or hMPV033/42C2_hlgG1 mixtures were mixed with 0.62 μl of chymotrypsin (Promega) with the ratio 1/200. The proteolytic mixtures were incubated overnight at 25° C.

### ASP-N Proteolysis

100 μl of the reduced/alkyled hMPV033/17D10 hlgG1, hMPV033/13E10_hlgG1 or hMPV033/42C2_hlgG1 mixtures were mixed with 0.62 μl of ASP-N(Promega) with the ratio 1/200. The proteolytic mixtures were incubated overnight at 37° C.

### Elastase Proteolysis

100 μl of the reduced/alkyled hMPV033/17D10 hlgG1, hMPV033/13E10_hlgG1 or hMPV033/42C2_hlgG1 mixtures were mixed with 1.24 μl of elastase (Promega) with the ratio 1/100. The proteolytic mixtures were incubated overnight at 37° C.

### Thermolysin Proteolysis

100 μl of the reduced/alkyled hMPV033/17D10 hlgG1, hMPV033/13E10_hlgG1 or hMPV033/42C2_hlgG1 mixtures were mixed with 2.48 μl of thermolysin (Promega) with a ratio 1/50. The proteolytic mixtures were incubated overnight at 70° C. After digestion formic acid 1% final was added to the solution.

The cross-linked peptides were analyzed using Xquest™ version 2.0 and Stavrox™ 3.6. software.

### Results

hMPV033/17D10_hlgG1

After Trypsin, Chymotrypsin, ASP-N, Elastase and Thermolysin proteolysis of the protein complex hMPV033/17D10_hlgG1 with deuterated d0d12, the nLC-Q-Exactive MS/MS analysis detected 12 cross-linked peptides between hMPV033 and 17D10_hlgG1. Using chemical cross-linking, High-Mass MALDI mass spectrometry, and nLC-Q-Exactive mass spectrometry, the molecular interface between hMPV033 and 17D10_hlgG1 was characterized (FIGS. 4A-4J). The analysis indicates that the interaction includes the following amino acids on hMPV033: 287, 293, 296, 364, 376, 417 and 419 of the hMPV033 amino acid sequence SEQ ID NO: 1.

hMPV033/13E10_hlgG1

After Trypsin, Chymotrypsin, ASP-N, Elastase and Thermolysin proteolysis of the protein complex hMPV033/13E10 hlgG1 with deuterated d0d12, the nLC-Q-Exactive MS/MS analysis detected 15 cross-linked peptides between hMPV033 and 13E10_hlgG1. Using chemical cross-linking, High-Mass MALDI mass spectrometry and nLC-Q-Exactive mass spectrometry the molecular interface between hMPV033 and 13E10 hlgG1 was characterized (FIG. 5A-5J). The analysis indicates that the interaction includes the following amino acids on hMPV033: 144, 160, 163, 188, 194 and 199 of the hMPV033 amino acid sequence SEQ ID NO: 1.

hMPV033/42C2_hlgG1

After Trypsin, Chymotrypsin, ASP-N, Elastase and Thermolysin proteolysis of the protein complex hMPV033/42C2 hlgG1 with deuterated d0d12, the nLC-Q-Exactive MS/MS analysis detected 15 cross-linked peptides between hMPV033 and 42C2_hlgG1. Using chemical cross-linking, High-Mass MALDI mass spectrometry and nLC-Q-Exactive mass spectrometry the molecular interface between hMPV033 and 42C2 hlgG1 was characterized (FIG. 6A-6J). The analysis indicates that the interaction includes the following amino acids on hMPV033: 44, 45, 49, 150, 156, 160, 229, 232 and 236 of the hMPV033 amino acid sequence SEQ ID NO: 1.

### Example 2: HMPV033 Competing Antibody ELISA

A competing antibody assay was developed to determine the titer of serum antibodies which can compete with mAb 17D10 hIgG1 for binding to the HMPV F protein.

| Abbreviation | Explanation |
|---|---|
| Ab | antibody |
| Bio | Biotinylated |
| ELISA | Enzyme-linked immunosorbent assay |
| F | Fusion protein |
| hMPV | Human metapneumovirus |
| HRP | Horseradish peroxidase |
| IgA | Immunoglobulin A |
| IgG | Immunoglobulin G |
| IgM | Immunoglobulin M |
| mAb | Monoclonal antibody |
| N/A | Not applicable |
| OD | Optical density |
| PBS | Phosphate buffered salt |
| QC | Quality control |
| SOP | Standard operating procedure |
| TBD | To be determined |
| TMB | 3,3′,5,5′-Tetramethylbenzidine |

### Assay Methods

Microtiter plates were coated with hMPV F antigen (dn5B-hMPV033). Serum samples were serially five-fold diluted after which an equal volume of a fixed concentration of biotinylated 17D10 hIgG1 antibody (mAb-Bio) was added. The mixture was added to the microtiter plates and incubated. As control, “17D10 hIgG1 mAb-Bio only” wells (maximal signal) and “sample dilution buffer only” wells (background signal) were analyzed. After incubation, unbound material is washed from the wells, and an HRP-conjugated streptavidin was added to all wells. The wells were then again washed to remove any unbound HRP-conjugated streptavidin. Next, the addition of TMB substrate triggered color development that is proportional to the amount of 17D10 hIgG1 mAb-Bio bound to the hMPV F antigen. Color development was quenched, and the optical density (OD450 nm and OD620 nm) was measured. The 17D10 hIgG1 mAb-Bio competing antibody binding titers are expressed as the reciprocal dilution at which 50% of the binding of 17D10 hIgG1 mAb-Bio is inhibited.

### Assay Optimization

Several hMPV F antigen coating concentrations are tested in combination with different concentrations of the 17D10 hIgG1 mAb-Bio and different dilutions of HRP-conjugated streptavidin. Maximum binding of 17D10 hIgG1 mAb-Bio to hMPV F protein results in an OD of ˜2.0 and low background OD.

### Example 3: Analysis of Antibody Binding to Pre- and Post-Fusion hMPV F Protein

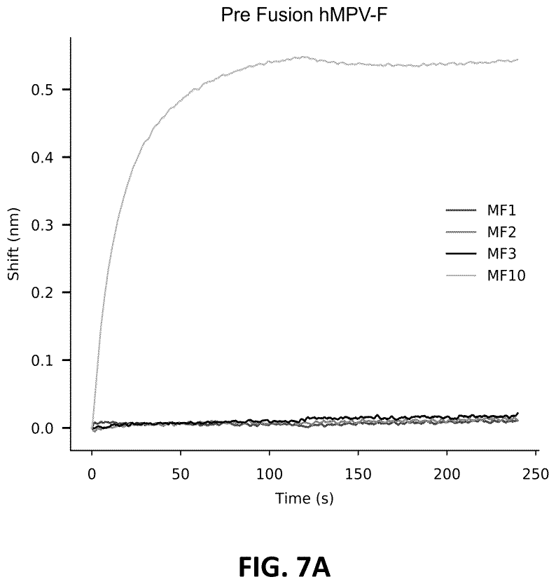

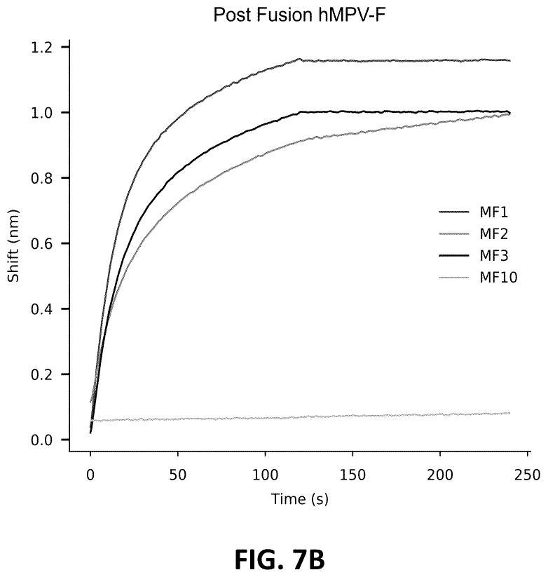

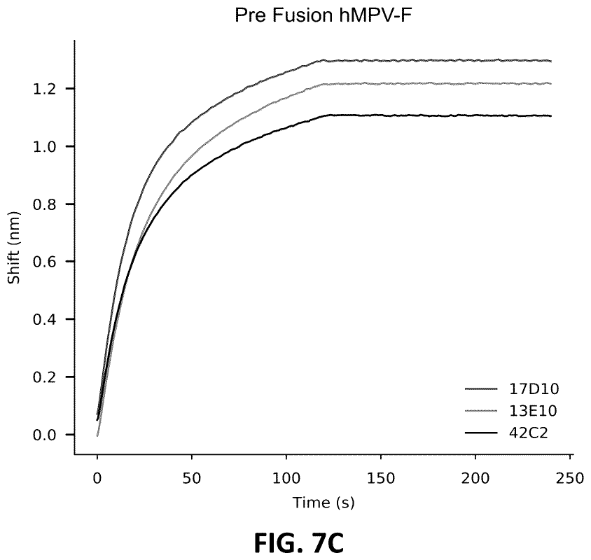

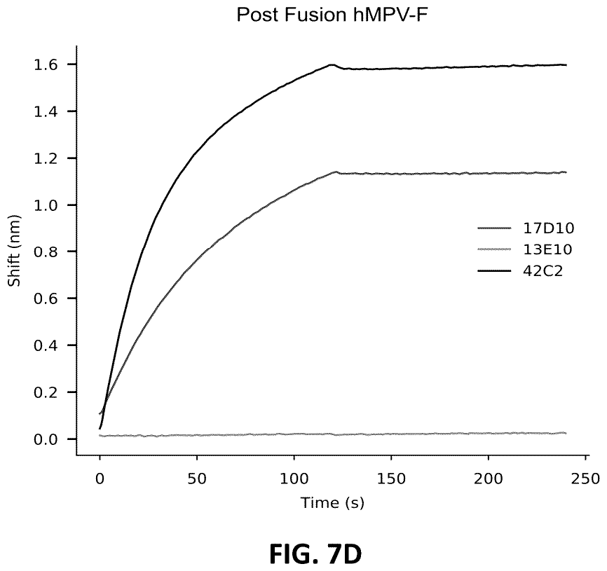

An CompA-hMPV033 (hMPV F protein) fusion protein, post-fusion hMPV F protein, and mouse antibodies of the present disclosure were normalized to a concentration of 10 μg/mL in BLI assay buffer (PBS, 0.5% BSA, 0.05% Tween 20, pH 7.4) and loaded at 200 μL per well of a black 96-well microplate. Protein G biosensors were hydrated before being dipped and baselined for 60 seconds in BLI assay buffer. The biosensors were then dipped into antibody wells for 120 seconds to immobilize the antibodies present but without reaching saturation, followed by an additional 60 second baseline step. The immobilized antibodies were allowed to associate with the antigens for 120 seconds before biosensors were dipped back into assay buffer for 120 seconds to observe dissociation. MF1, MF2, and MF3 are post-fusion hMPV F protein specific antibodies and MF10 is a pre-fusion hMPV F protein specific antibody that were used as controls. Binding of the control antibodies to pre- and post-fusion hMPV F protein is shown in FIGS. 7A and 7B. Binding of 17D10, 42C2 and 13E10 antibodies to both hMPV F protein conformations was observed and 17D10 and 42C2 antibodies bound both pre- and post-fusion hMPV F protein and 13E10 antibody bound only pre-fusion hMPV F protein as shown in FIGS. 7C and 7D.

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. (canceled)

2. An antigen-binding molecule specific to a human metapneumovirus (hMPV) F protein, comprising: a heavy chain and a light chain,
wherein the heavy chain comprises an H-CDR1, an H-CDR2, and an H-CDR3 wherein: H-CDR1 comprises the sequence GYTFTSY (SEQ ID NO: 3); H-CDR2 comprises the sequence YPGSGS (SEQ ID NO: 4); and H-CDR3 comprises the sequence LLRLTFDV (SEQ ID NO: 5); and
wherein the light chain comprises an L-CDR1, an L-CDR2, and an L-CDR3, wherein: L-CDR1 comprises the sequence RASQDISNYLN (SEQ ID NO: 7); L-CDR2 comprises the sequence YTSGLHS (SEQ ID NO: 8); and L-CDR3 comprises the sequence QQGNTLPWT (SEQ ID NO: 9).

3.-7. (canceled)

8. The antigen-binding molecule of claim 2,
wherein the heavy chain comprises a sequence having at least 70%, at least 80%, at least 90%, at least 95%, at least 97%, at least 98%, at least 99%, or 100% sequence identity to SEQ ID NO: 2, and/or
wherein the light chain comprises a sequence having at least 70%, at least 80%, at least 90%, at least 95%, at least 97%, at least 98%, at least 99%, or 100% sequence identity to SEQ ID NO: 6.

9. The antigen-binding molecule of claim 2, comprising
a variable heavy (VH) chain domain wherein the VH domain comprises a sequence having at least 70%, at least 80%, at least 90%, at least 95%, at least 97%, at least 98%, at least 99%, or 100% sequence identity to SEQ ID NO: 50, and
a variable light (VL) chain domain wherein the VL domain comprises a sequence having at least 70%, at least 80%, at least 90%, at least 95%, at least 97%, at least 98%, at least 99%, or 100% sequence identity to SEQ ID NO: 51.

10. (canceled)

11. An antigen-binding molecule specific to a human metapneumovirus (hMPV) F protein, comprising: a heavy chain and a light chain,
wherein the heavy chain comprises an H-CDR1, an H-CDR2, and an H-CDR3 wherein: H-CDR1 comprises the sequence GFTFTDY (SEQ ID NO: 11); H-CDR2 comprises the sequence RNKDNGYT (SEQ ID NO: 12); and H-CDR3 comprises the sequence YYFGYDGDYFDY (SEQ ID NO: 13); and
wherein the light chain comprises an L-CDR1, an L-CDR2, and an L-CDR3, wherein: L-CDR1 comprises the sequence SASSSISSNYLH (SEQ ID NO: 15); L-CDR2 comprises the sequence RTSNLAS (SEQ ID NO: 16); and L-CDR3 comprises the sequence QQGSSLPRT (SEQ ID NO: 17).

12.-13. (canceled)

14. The antigen-binding molecule of claim 11,
wherein the heavy chain comprises a sequence having at least 70%, at least 80%, at least 90%, at least 95%, at least 97%, at least 98%, at least 99%, or 100% sequence identity to SEQ ID NO: 10, and/or
wherein the light chain comprises a sequence having at least 70%, at least 80%, at least 90%, at least 95%, at least 97%, at least 98%, at least 99%, or 100% sequence identity to SEQ ID NO: 14.

15. The antigen-binding molecule of claim 11, comprising
a variable heavy (VH) chain domain wherein the VH domain comprises a sequence having at least 70%, at least 80%, at least 90%, at least 95%, at least 97%, at least 98%, at least 99%, or 100% sequence identity to SEQ ID NO: 52, and
a variable light (VL) chain domain wherein the VL domain comprises a sequence having at least 70%, at least 80%, at least 90%, at least 95%, at least 97%, at least 98%, at least 99%, or 100% sequence identity to SEQ ID NO: 53.

16. (canceled)

17. An antigen-binding molecule specific to a human metapneumovirus (hMPV) F protein, comprising: a heavy chain and a light chain,
wherein the heavy chain comprises an H-CDR1, an H-CDR2, and an H-CDR3 wherein: H-CDR1 comprises the sequence GFSLSTFGM (SEQ ID NO: 19); H-CDR2 comprises the sequence WWDDD (SEQ ID NO: 20); and H-CDR3 comprises the sequence IVKVLEQYFDV (SEQ ID NO: 21); and
wherein the light chain comprises an L-CDR1, an L-CDR2, and an L-CDR3, wherein: L-CDR1 comprises the sequence KASQDVGTAVA (SEQ ID NO: 23); L-CDR2 comprises the sequence WASTRHT (SEQ ID NO: 24); and L-CDR3 comprises the sequence QQYTSYPLT (SEQ ID NO: 25).

18.-19. (canceled)

20. The antigen-binding molecule of claim 17,
wherein the heavy chain comprises a sequence having at least 70%, at least 80%, at least 90%, at least 95%, at least 97%, at least 98%, at least 99%, or 100% sequence identity to SEQ ID NO: 18, and/or
wherein the light chain comprises a sequence having at least 70%, at least 80%, at least 90%, at least 95%, at least 97%, at least 98%, at least 99%, or 100% sequence identity to SEQ ID NO: 22.

21. The antigen-binding molecule of claim 17, comprising
a variable heavy (VH) chain domain wherein the VH domain comprises a sequence having at least 70%, at least 80%, at least 90%, at least 95%, at least 97%, at least 98%, at least 99%, or 100% sequence identity to SEQ ID NO: 54, and
a variable light (VL) chain domain wherein the VL domain comprises a sequence having at least 70%, at least 80%, at least 90%, at least 95%, at least 97%, at least 98%, at least 99%, or 100% sequence identity to SEQ ID NO: 55.

22. The antigen-binding molecule of claim 2, wherein the antigen-binding molecule is an immunoglobulin molecule.

23. The antigen-binding molecule of claim 22, wherein the immunoglobulin is an IgG1, IgG2, IgG3, or IgG4 molecule.

24. The antigen-binding molecule of claim 22, wherein the immunoglobulin is a humanized antibody.

25. The antigen-binding molecule of claim 22, wherein the immunoglobulin is a neutralizing antibody.

26. The antigen-binding molecule of claim 2, wherein antigen-binding molecule specifically binds an hMPV F protein in the pre-fusion conformation.

27. The antigen-binding molecule of claim 2, wherein antigen-binding molecule binds an hMPV F protein in the pre-fusion or post-fusion conformation.

28. A polynucleotide encoding the antigen-binding molecule of claim 2.

29. A pharmaceutical composition or vaccine, comprising the antigen-binding molecule of claim 2, and a pharmaceutically acceptable carrier, diluent, or excipient.

30.-36. (canceled)

37. A method of treating or preventing an hMPV infection in a subject in need thereof, wherein the method comprises administering to the subject an effective amount of the pharmaceutical composition or vaccine of claim 29.

38.-40. (canceled)

41. The method of claim 37, wherein the subject is an elderly subject.

42. The method of claim 37, wherein the antigen-pharmaceutical composition or vaccine is administered by intramuscular injection, intravenous injection, or subcutaneous injection.

43.-44. (canceled)
